(19) United States
(12) Patent Application Publication
(10) Pub. No.: US 2025/0066356 A1
(43) Pub. Date: Feb. 27, 2025

(54) LRRK2 INHIBITORS

(71) Applicant: INTERLINE THERAPEUTICS, INC., Brisbane, CA (US)

(72) Inventors: Robert J. MOREAU, Walnut Creek, CA (US); David C. TULLY, Danville, CA (US); Zuojun GUO, Pleasanton, CA (US); Mario G. CARDOZO, San Francisco, CA (US); Zachary K. SWEENEY, Redwood City, CA (US)

(21) Appl. No.: 18/941,521

(22) Filed: Nov. 8, 2024

Related U.S. Application Data
(63) Continuation of application No. PCT/US2023/021836, filed on May 11, 2023.
(60) Provisional application No. 63/341,065, filed on May 12, 2022.

(51) Int. Cl. C07D 471/04; A61K 31/4745

(52) U.S. Cl. CPC C07D 471/04; A61K 31/4745

(57) ABSTRACT

The present invention relates to imidazo[4,5-c]quinoline compounds of Formula (I), and pharmaceutically acceptable salts thereof. The invention is also directed to pharmaceutical compositions comprising the compounds of Formula (I) and to use of the compounds in the treatment of diseases associated with LRRK2, such as neurodegenerative diseases including Parkinson's disease or Alzheimer's disease, or inflammatory bowel disease such as Crohn's disease.

### CROSS-REFERENCES TO RELATED APPLICATIONS

This application claims the benefit under 35 U.S.C. § 120 of PCT Appl. No. PCT/US2023/021836, filed May 11, 2023, which claims priority to U.S. Provisional Application No. 63/341,065, filed May 12, 2022, each of which is incorporated by reference herein in its entirety for all purposes.

### BACKGROUND OF THE INVENTION

LRRK2 is a 286 kDa protein in the ROCO protein family with a complex multidomain structure. Protein motifs that have been established for LRRK2 include an armadillo-like (ARM) domain, an ankyrin-like (ANK) domain, a leucine-rich repeat (LRR) domain, a Ras (renin-angiotensin system) of complex (ROC) domain, a C-terminal of ROC (COR) domain, a kinase domain, and a C-terminal WD40 domain. The ROC domain binds guanosine triphosphate (GTP) and the COR domain may be a regulator of the ROC domain's GTPase activity. The kinase domain has structural homology to the MAP kinase kinase kinases (MAPKKK) and has been shown to phosphorylate a number of cellular proteins in vitro, but the endogenous substrate has yet to be determined. LRRK2 has been found in various regions of the brain as well as in a number of peripheral tissues including heart, lung, spleen, and kidney.

LRRK2 can play a complex role in multiple cellular processes as a consequence of its multi-domain construct, each associated with putative protein-protein interactions, guanosine triphosphatase (GTPase) activity, and kinase activity. For example, LRRK2 has been associated with NFAT inhibition in the immune system and has been linked to vesicle trafficking, presynaptic homeostasis, mammalian target of rapamycin (mTOR) signaling, signaling through the receptor tyrosine kinase MET in papillary renal and thyroid carcinomas, cytoskeletal dynamics, the mitogen-activated protein kinase (MAPK) pathway, the tumor necrosis factor-α (TNF-α) pathway, the Wnt pathway and autophagy. Genome-wide association (GWA) genetic studies have implicated LRRK2 in the pathogenesis of various human diseases such as PD and inflammatory bowel disease (such as Crohn's disease) (Lewis, P. A. and Manzoni, C. Science Signaling 2012, 5(207), pe2).

Parkinson's disease (PD) is a relatively common age-related neurodegenerative disorder resulting from the progressive loss of dopamine-producing neurons and which affects up to 4% of the population over age 80. PD is characterized by both motor symptoms, such as tremor at rest, rigidity, akinesia and postural instability as well as non-motor symptoms such as impairment of cognition, sleep and sense of smell. GWA studies have linked LRRK2 to PD and many patients with point mutations in LRRK2 present symptoms that are indistinguishable from those with idiopathic PD. Over 20 LRRK2 mutations have been associated with autosomal-dominant Parkinsonism, and the R1441C, R1441G, R1441H, Y1699C, G2019S, 12020T and N1437H missense mutations are considered to be pathogenic. The LRRK2 R1441G mutation has been shown to increase the release of proinflammatory cytokines (higher levels of TNF-α, IL-10, IL-12 and lower levels of IL-10) in microglial cells from transgenic mice and thus may result in direct toxicity to neurons (Gillardon, F. et al. Neuroscience 2012, 208, 41-48). In a murine model of neuroinflammation, induction of LRRK2 in microglia was observed and inhibition of LRRK2 kinase activity with small molecule LRRK2 inhibitors (LRRK2-IN-1 or sunitinib) or LRRK2 knockout resulted in attenuation of TNF-α secretion and nitric oxide synthase (iNOS) induction (Moehle, M. et al. J. Neurosci. 2012, 32(5), 1602-1611). The most common of the LRRK2 mutations, G2019S, is present in more than 85% of PD patients carrying LRRK2 mutations. This mutation, which is present in the LRRK2 kinase domain, leads to an enhancement of LRRK2 kinase activity. In the human brain LRRK2 expression is highest in the same regions of the brain that are impacted by PD, and LRRK2 is found in Lewy Bodies, a hallmark of PD. Recent studies indicate that a potent, selective, brain-penetrant kinase inhibitor for LRRK2 could be a therapeutic treatment for PD.

Dementia results from a wide variety of distinctive pathological processes. The most common pathological processes causing dementia are Alzheimer's disease (AD), cerebral amyloid angiopathy (CM) and prion-mediated diseases (see, e.g., Haan et al., Clin. Neurol. Neurosurg. 1990, 92(4):305-310; Glenner et al., J. Neurol. Sci. 1989, 94:1-28). AD is a progressive, neurodegenerative disorder characterized by memory impairment and cognitive dysfunction. AD affects nearly half of all people past the age of 85, the most rapidly growing portion of the United States population. As such, the number of AD patients in the United States is expected to increase from about 4 million to about 14 million by 2050. LRRK2 mutations have been associated with AD-like pathology, which suggests that there may be a partial overlap between the neurodegenerative pathways in both AD and PD (Zimprach, A. et al. Neuron 2004, 44, 601-607). In addition, the LRRK2 R1628P variant (COR domain) has been associated with an increased incidence of AD in a certain population, perhaps resulting from increased apoptosis and cell death (Zhao, Y. et al.; Neurobiology of Aging 2011, 32, 1990-1993).

Inflammatory bowel disease (IBD), such as ulcerative colitis or Crohn's disease (CD), is a complex disease that are believed to result from an inappropriate immune response to microbiota in the intestinal tract. Genome wide association studies have recently identified LRRK2 as a major susceptibility gene for Crohn's disease, particularly the M2397T polymorphism in the WD40 domain (Liu, Z. et al. Nat. Immunol. 2011, 12, 1063-1070). LRRK2 deficient mice were found to be more susceptible to dextran sodium sulfate induced colitis than their wild-type counterparts, indicating that LRRK2 may play a role in the pathogenesis of IBD (Liu, Z. and Lenardo, M.; Cell Research 2012, 1-3).

Both non-selective and selective small molecule compounds with LRRK2 inhibitory activity such as staurosporine, sunitinib, LRRK2-IN-1, CZC-25146, TAE684 and those in WO 2011/141756, WO 2012/028629, WO 2012/058193, WO 2017/046675, WO 2018/163030, WO 2018/163066, WO 2021/080929, and US 20210002260 have been described. It is desirable to provide compounds which are potent and selective inhibitors of LRRK2 with a favorable pharmacokinetic profile and the ability to traverse the blood brain barrier. The present invention is directed to addressing these problems and others.

### BRIEF SUMMARY OF THE INVENTION

In some embodiments, the present disclosure provides a compound of the present invention is a compound of Formula (I):

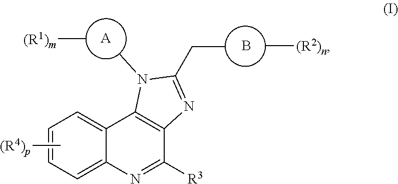

or a pharmaceutically acceptable salt thereof, wherein
Ring A is a 5 to 6 membered heterocycloalkyl having 1 to 2 heteroatoms each independently N, O or S, or a 5 to 6 membered heteroaryl having 1 or 2 heteroatoms each independently N, O or S;
each R¹ is independently C₁₋₆ alkyl or ═O;
Ring B is a 5 to 6 membered heteroaryl having 1 to 4 heteroatoms each independently N, O or S;
each R² is a C₁₋₆ alkyl, —OH, ═O, C(O)R^{2a}, C(O)OR^{2b}, OC(O)R^{2a}, S(O)₂R^{2a}, S(O)₂OR^{2b}, OS(O)₂R^{2a}, N(R^{2b})S(O)₂R^{2a}, S(O)₂N(R^{2b})(R^{2c}), C₃₋₆ cycloalkyl, C₁₋₆ alkyl-C₃₋₆ cycloalkyl, or 3 to 6 membered heterocycloalkyl having 1 to 3 heteroatoms each independently N, O or S, wherein each alkyl or cycloalkyl is substituted with 1 to 3 R^{2d} groups, and wherein each heterocycloalkyl is substituted with 0 to 3 R^{2e} groups;
each R^{2b} and R^{2c} is hydrogen or C₁₋₆ alkyl;
each R^{2d} is independently C(O)R^{2d1} or S(O)₂R^{2d1};
each R^{2e} is independently C₁₋₆ alkyl, —OH, ═O, C(O)R^{2e1} or S(O)₂R^{2e1};
each R^{2a}, R^{2d1} and R^{2e1} is independently C₁₋₆ alkyl;
each R³ and R⁴ is hydrogen, C₁₋₆ alkyl, C₁₋₆ alkoxy, halogen, C₁₋₆ haloalkyl, C₁₋₆ haloalkoxy, or —CN;
subscript n is 0, 1 or 2; and
subscript m and p are each independently an integer from 1 to 4.

In some embodiments, the present disclosure provides a pharmaceutical composition of the invention comprises a compound of the disclosure, or pharmaceutically acceptable salt thereof, and a pharmaceutically acceptable excipient.

In some embodiments, the present disclosure provides a method of inhibiting LRRK2 in a cell comprises contacting the cell with an effective amount of a compound of the disclosure, or pharmaceutically acceptable salt thereof.

In some embodiments, the present disclosure provides a method of treating a LRRK2-associated disease or condition in a subject in need thereof comprises administering to the subject a therapeutically effective amount of a compound of the disclosure, or pharmaceutically acceptable salt thereof.

### DETAILED DESCRIPTION OF THE INVENTION

### I. General

The compounds of the present disclosure include compounds of Formula (I), (Ia), (Ib), and (Ic), including the compounds of the Examples. These compounds are useful for inhibiting LRRK2, as well as for treating LRRK2-mediated diseases such as, but not limited to, Parkinson's disease, Lewy body dementia, frontotemporal dementia, corticobasal dementia, progressive supranuclear palsy, Alzheimer's disease, tauopathy disease, or alpha-synucleinopathy.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

“A,” “an,” or “the” refers to not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“About” when referring to a value includes the stated value +/−10% of the stated value. For example, about 50% includes a range of from 45% to 55%, while about 20 molar equivalents includes a range of from 18 to 22 molar equivalents. Accordingly, when referring to a range, “about” refers to each of the stated values +/−10% of the stated value of each end of the range. For instance, a ratio of from about 1 to about 3 (weight/weight) includes a range of from 0.9 to 3.3.

“Alkyl” is a linear or branched saturated monovalent hydrocarbon. An alkyl group can have 1 to 18 carbon atoms (i.e., C₁₋₁₈ alkyl) or 1 to 8 carbon atoms (i.e., C₁₋₈ alkyl) or 1 to 6 carbon atoms (i.e., C₁₋₆ alkyl) or 1 to 4 carbon atoms (i.e., C₁₋₄ alkyl). Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃. Other alkyl groups include heptyl, octyl, nonyl, decyl, undecyl, dodecyl, pentadecyl, hexadecyl, heptadecyl and octadecyl. Alkyl groups can be substituted or unsubstituted.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

“Halo” or “halogen” as used herein refers to fluoro (—F), chloro (—Cl), bromo (—Br) and iodo (—I).

An “oxo” substituent refers to the divalent substitution “═O” present on a single atom. For example, an oxo substitution combined with the carbon to which it is attached is a carbonyl (C═O).

“Haloalkyl” as used herein refers to an alkyl as defined herein, wherein one or more hydrogen atoms of the alkyl are independently replaced by a halo substituent, which may be the same or different. For example, C₁₋₄ haloalkyl is a C₁₋₄ alkyl wherein one or more of the hydrogen atoms of the C₁₋₄ alkyl have been replaced by a halo substituent. Examples of haloalkyl groups include but are not limited to fluoromethyl, fluorochloromethyl, difluoromethyl, difluorochloromethyl, trifluoromethyl, 1,1,1-trifluoroethyl and pentafluoroethyl. Haloalkyl groups can be substituted or unsubstituted.

“Haloalkoxy” refers to an alkoxy group where some or all of the hydrogen atoms are substituted with halogen atoms. As for an alkyl group, haloalkoxy groups can have any suitable number of carbon atoms, such as C₁-6. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc. Haloalkoxy groups can be substituted or unsubstituted.

“Cycloalkyl” refers to a single saturated or partially unsaturated all carbon ring having 3 to 20 annular carbon atoms (i.e., C₃₋₂₀ cycloalkyl), for example from 3 to 12 annular atoms, for example from 3 to 10 annular atoms, or 3 to 8 annular atoms, or 3 to 6 annular atoms, or 3 to 5 annular atoms, or 3 to 4 annular atoms. The term “cycloalkyl” also includes multiple condensed, saturated and partially unsaturated all carbon ring systems (e.g., ring systems comprising 2, 3 or 4 carbocyclic rings). Accordingly, cycloalkyl includes multicyclic carbocycles such as a bicyclic carbocycles (e.g., bicyclic carbocycles having 6 to 12 annular carbon atoms such as bicyclo[3.1.0]hexane and bicyclo[2.1.1]hexane), and polycyclic carbocycles (e.g., tricyclic and tetracyclic carbocycles with up to 20 annular carbon atoms). The rings of a multiple condensed ring system can be connected to each other via fused, spiro and bridged bonds when allowed by valency requirements. Non-limiting examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl and 1-cyclohex-3-enyl. Cycloalkyl groups can be substituted or unsubstituted.

“Alkyl-cycloalkyl” refers to a radical having an alkyl component and a cycloalkyl component, where the alkyl component links the cycloalkyl component to the point of attachment. The alkyl component is as defined above, except that the alkyl component is at least divalent, that is, an alkylene, to link to the cycloalkyl component and to the point of attachment. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The cycloalkyl component is as defined within. Exemplary alkyl-cycloalkyl groups include, but are not limited to, methyl-cyclopropyl, methyl-cyclobutyl, methyl-cyclopentyl and methyl-cyclohexyl. Alkyl-cycloalkyl groups can be substituted or unsubstituted.

“Heterocyclyl” or “heterocycle” or “heterocycloalkyl” as used herein refers to a single saturated or partially unsaturated non-aromatic ring or a multiple ring system having at least one heteroatom in the ring (i.e., at least one annular heteroatom selected from oxygen, nitrogen, and sulfur) wherein the multiple ring system includes at least non-aromatic ring containing at least one heteroatom. The multiple ring system can also include other aromatic rings and non-aromatic rings. Unless otherwise specified, a heterocyclyl group has from 3 to 20 annular atoms, for example from 3 to 12 annular atoms, for example from 3 to 10 annular atoms, or 3 to 8 annular atoms, or 3 to 6 annular atoms, or 3 to 5 annular atoms, or 4 to 6 annular atoms, or 4 to 5 annular atoms. Thus, the term includes single saturated or partially unsaturated rings (e.g., 3, 4, 5, 6 or 7-membered rings) having from 1 to 6 annular carbon atoms and from 1 to 3 annular heteroatoms selected from the group consisting of oxygen, nitrogen and sulfur in the ring. The heteroatoms can optionally be oxidized to form —N(—OH)—, ═N(—O—)—, —S(═O)— or —S(═O)₂—. The rings of the multiple condensed ring (e.g. bicyclic heterocyclyl) system can be connected to each other via fused, spiro and bridged bonds when allowed by valency requirements. Heterocycles include, but are not limited to, azetidine, aziridine, imidazolidine, morpholine, oxirane (epoxide), oxetane, thietane, piperazine, piperidine, pyrazolidine, piperidine, pyrrolidine, tetrahydrofuran, tetrahydrothiophene, dihydropyridine, tetrahydropyridine, quinuclidine, 2-oxa-6-azaspiro[3.3]heptan-6-yl, 6-oxa-1-azaspiro[3.3]heptan-1-yl, 2-thia-6-azaspiro[3.3]heptan-6-yl, 2,6-diazaspiro[3.3]heptan-2-yl, 2-azabicyclo[3.1.0]hexan-2-yl, 3-azabicyclo[3.1.0]hexanyl, 2-azabicyclo[2.1.1]hexanyl, 2-azabicyclo[2.2.1]heptan-2-yl, 4-azaspiro[2.4]heptanyl, 5-azaspiro[2.4]heptanyl, and the like. Heterocycloalkyl groups can be substituted or unsubstituted.

Heterocycloalkyl rings also include 9 to 15 membered fused ring heterocycloalkyls having 2, 3, or more rings wherein at least one ring is an aryl ring and at least one ring is a non-aromatic ring containing at least one heteroatom. Representative fused bicyclic heterocycloalkyls include, but are not limited to, indoline (dihydroindole), isoindoline (dihydroisoindole), indazoline (dihydroindazole), benzo[d]imidazole, dihydroquinoline, dihydroisoquinoline, dihydrobenzofuran, dihydroisobenzofuran, benzo[d][1,3]dioxol, dihydrobenzo[b]dioxine, dihydrobenzo[d]oxazole, dihydrobenzo[b]thiophene, dihydroisobenzo[c]thiophene, dihydrobenzo[d]thiazole, dihydrobenzo[c]isothiazole, and benzo[b][1,4]thiazine, as shown in the structures below:

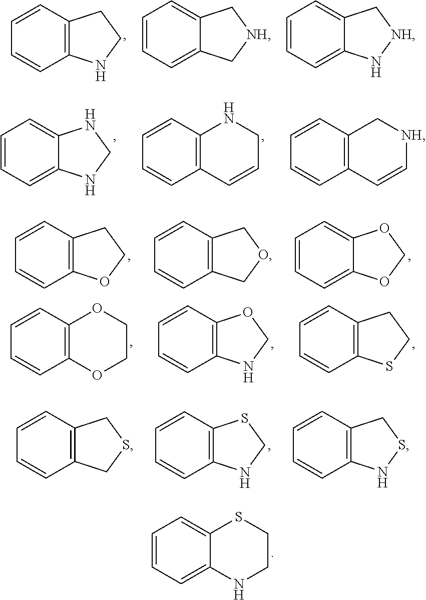

Fused bicyclic heterocycloalkyls can also be represented by the following structure:

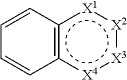

wherein X¹, X², X³ and X⁴ are each independently absent, —CH₂—, —NH—, —O— or —S—, at least one of X¹, X², X³ and X⁴ is —NH—, —O— or —S—, and the dashed circle represents a saturated or partially unsaturated non-aromatic ring. The fused bicyclic heterocycloalkyls can be substituted or unsubstituted.

“Alkyl-heterocycloalkyl” refers to a radical having an alkyl component and a heterocycloalkyl component, where the alkyl component links the heterocycloalkyl component to the point of attachment. The alkyl component is as defined above, except that the alkyl component is at least divalent, an alkylene, to link to the heterocycloalkyl component and to the point of attachment. The alkyl component can include any number of carbons, such as C₁₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. The heterocycloalkyl component is as defined above. Alkyl-heterocycloalkyl groups can be substituted or unsubstituted. Alkyl-heterocycloalkyl groups can be substituted or unsubstituted.

“Aryl” as used herein refers to a single all carbon aromatic ring or a multiple condensed all carbon ring system wherein at least one of the rings is aromatic. For example, in some embodiments, an aryl group has 6 to 20 carbon atoms, 6 to 14 carbon atoms, or 6 to 12 carbon atoms. Aryl includes a phenyl radical. Aryl also includes multiple condensed ring systems (e.g., ring systems comprising 2, 3 or 4 rings) having 9 to 20 carbon atoms, e.g., 9 to 16 carbon atoms, in which at least one ring is aromatic and wherein the other rings may be aromatic or not aromatic (i.e., carbocycle). Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is also to be understood that when reference is made to a certain atom-range membered aryl (e.g., 6-10 membered aryl), the atom range is for the total ring atoms of the aryl. For example, a 6-membered aryl would include phenyl and a 10-membered aryl would include naphthyl and 1,2,3,4-tetrahydronaphthyl. Non-limiting examples of aryl groups include, but are not limited to, phenyl, indenyl, naphthyl, 1,2,3,4-tetrahydronaphthyl, anthracenyl, and the like. Aryl groups can be substituted or unsubstituted.

“Alkyl-aryl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₁₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The aryl component is as defined above. Examples of alkyl-aryl groups include, but are not limited to, benzyl and ethyl-benzene. Alkyl-aryl groups can be substituted or unsubstituted.

“Heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from 1 to 6 carbon atoms and 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from heteroaryls (to form for example 1,8-naphthyridinyl), heterocycles, (to form for example 1,2,3,4-tetrahydro-1,8-naphthyridinyl), carbocycles (to form for example 5,6,7,8-tetrahydroquinolyl) and aryls (to form for example indazolyl) to form the multiple condensed ring system. Thus, a heteroaryl (a single aromatic ring or multiple condensed ring system) has 1-20 carbon atoms and 1-6 heteroatoms within the heteroaryl ring. Such multiple condensed ring systems may be optionally substituted with one or more (e.g., 1, 2, 3 or 4) oxo groups on the carbocycle or heterocycle portions of the condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). It also to be understood that when a reference is made to a certain atom-range membered heteroaryl (e.g., a 5 to 10 membered heteroaryl), the atom range is for the total ring atoms of the heteroaryl and includes carbon atoms and heteroatoms. For example, a 5-membered heteroaryl would include a thiazolyl and a 10-membered heteroaryl would include a quinolinyl. Exemplary heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thionaphthenyl, pyrrolo[2,3-b]pyridinyl, quinazolinyl-4(3H)-one, and triazolyl. Heteroaryl groups can be substituted or unsubstituted.

“Alkyl-heteroaryl” refers to a radical having an alkyl component and a heteroaryl component, where the alkyl component links the heteroaryl component to the point of attachment. The alkyl component is as defined above, except that the alkyl component is at least divalent, an alkylene, to link to the heteroaryl component and to the point of attachment. The alkyl component can include any number of carbons, such as C₁₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heteroaryl component is as defined within. Alkyl-heteroaryl groups can be substituted or unsubstituted.

A “compound of the present disclosure” includes compounds disclosed herein, for example a compound of the present disclosure includes compounds of Formula (I), (Ia), (Ib), and (Ic), including the compounds of the Examples.

Pharmaceutically acceptable salts, tautomeric forms, and polymorphs of the compounds are also described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts.

Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

“Racemates” refers to a mixture of enantiomers. The mixture can comprise equal or unequal amounts of each enantiomer.

“Stereoisomer” and “stereoisomers” refer to compounds that differ in the chirality of one or more stereocenters. Stereoisomers include enantiomers and diastereomers. The compounds may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of Advanced Organic Chemistry, 4th ed., J. March, John Wiley and Sons, New York, 1992).

“Tautomer” refers to alternate forms of a compound that differ in the position of a proton, such as enol-keto and imine-enamine tautomers, or the tautomeric forms of heteroaryl groups containing a ring atom attached to both a ring —NH— and a ring ═N— such as pyrazoles, imidazoles, benzimidazoles, triazoles, and tetrazoles.

Unless defined otherwise, all technical and scientific terms used herein have the same meaning as commonly understood by one of ordinary skill in the art. A dash at the front or end of a chemical group is a matter of convenience; chemical groups may be depicted with or without one or more dashes without losing their ordinary meaning. A wavy line drawn through a line in a structure indicates a point of attachment of a group. A dashed line indicates an optional bond. Unless chemically or structurally required, no directionality is indicated or implied by the order in which a chemical group is written or the point at which it is attached to the remainder of the molecule. For instance, the group “—SO₂CH₂—” is equivalent to “—CH₂SO₂—” and both may be connected in either direction. Similarly, an “arylalkyl” group, for example, may be attached to the remainder of the molecule at either an aryl or an alkyl portion of the group. A prefix such as “Cᵤ₋ᵥ” or (Cᵤ-Cᵥ) indicates that the following group has from u to v carbon atoms. For example, “C₁₋₆alkyl” and “C₁-C₆ alkyl” both indicate that the alkyl group has from 1 to 6 carbon atoms.

“Composition” as used herein is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

“Pharmaceutically effective amount” refers to an amount of a compound of the present disclosure in a formulation or combination thereof, that provides the desired therapeutic or pharmaceutical result.

“Pharmaceutical composition” as used herein refers to a product comprising the specified ingredients in the specified amounts, as well as any product, which results, directly or indirectly, from combination of the specified ingredients in the specified amounts. The pharmaceutical composition is generally safe for biological use.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Inhibit” or “inhibiting” LRRK2 as used herein refers to reducing the activity and/or function of LRRK2 enzyme. LRRK2 enzyme activity can be measured by any assay method known in the art, including an assay described in WO 2011/141756, WO 2012/028629, WO 2012/058193, WO 2017/046675, WO 2018/163030, WO 2018/163066, WO 2021/080929, or US 20210002260, or an assay described herein, such as an assay found in the Examples.

“Treatment” or “treat” or “treating” as used herein refers to an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In some embodiments, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival.

“Therapeutically effective amount” or “effective amount” as used herein refers to an amount that is effective to elicit the desired biological or medical response, including the amount of a compound that, when administered to a subject for treating a disease, is sufficient to effect such treatment for the disease. The effective amount can vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject. The administration can be carried out according to a schedule specifying frequency of administration, dose for administration, and other factors.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present disclosure is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present disclosure within seconds or minutes. In some embodiments, a unit dose of a compound of the present disclosure is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present disclosure. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the patient.

“Subject” refers to animals such as mammals, including, but not limited to, primates (e.g., humans), cows, sheep, goats, horses, dogs, cats, rabbits, rats, mice and the like. In some embodiments, the subject is a human.

“Disease” or “condition” refer to a state of being or health status of a patient or subject capable of being treated with a compound, pharmaceutical composition, or method provided herein.

### III. Compounds

The compounds of the present disclosure include compounds of Formula (I), (Ia), (Ib), and (Ic), including the compounds of the Examples.

In some embodiments, a compound of the present invention is a compound of Formula (I):

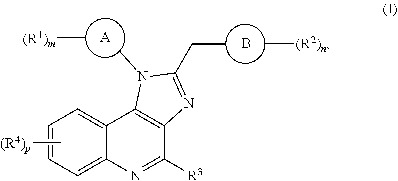

or a pharmaceutically acceptable salt thereof, wherein
Ring A is a 5 to 6 membered heterocycloalkyl having 1 to 2 heteroatoms each independently N, O or S, or a 5 to 6 membered heteroaryl having 1 or 2 heteroatoms each independently N, O or S;
each R¹ is independently C₁₋₆ alkyl or ═O;
Ring B is a 5 to 6 membered heteroaryl having 1 to 4 heteroatoms each independently N, O or S;
each R² is a C₁₋₆ alkyl, —OH, ═O, C(O)R^{2a}, C(O)OR^{2b}, OC(O)R^{2a}, S(O)₂R^{2a}, S(O)₂OR^{2b}, OS(O)₂R^{2a}, N(R^{2b})S(O)₂R^{2a}, S(O)₂N(R^{2b})(R^{2c}), C₃₋₆ cycloalkyl, C₁₋₆ alkyl-C₃₋₆ cycloalkyl, or 3 to 6 membered heterocycloalkyl having 1 to 3 heteroatoms each independently N, O or S, wherein each alkyl or cycloalkyl is substituted with 1 to 3 R^{2d} groups, and wherein each heterocycloalkyl is substituted with 0 to 3 R^{2e} groups;
each R^{2b} and R^{2c} is hydrogen or C₁₋₆ alkyl;
each R^{2d} is independently C(O)R^{2d1} or S(O)₂R^{2d1};
each R^{2e} is independently C₁₋₆ alkyl, —OH, ═O, C(O)R^{2e1} or S(O)₂R^{2e1};
each R^{2a}, R^{2d1} and R^{2e1} is independently C₁₋₆ alkyl;
each R³ and R⁴ is hydrogen, C₁₋₆ alkyl, C₁₋₆ alkoxy, halogen, C₁₋₆ haloalkyl, C₁₋₆ haloalkoxy, or —CN;
subscript n is 0, 1 or 2; and
subscript m and p are each independently an integer from 1 to 4.

In some embodiments, the compound or pharmaceutically acceptable salt thereof, is the compound wherein subscript n is 1 or 2. In some embodiments, the compound or pharmaceutically acceptable salt thereof, is the compound wherein subscript n is 1.

In some embodiments, the compound or pharmaceutically acceptable salt thereof, is the compound wherein subscript m is 1, 2, 3, or 4. In some embodiments, the compound or pharmaceutically acceptable salt thereof, is the compound wherein subscript m is 1 or 2. In some embodiments, the compound or pharmaceutically acceptable salt thereof, is the compound wherein subscript m is 1.

In some embodiments, the compound or pharmaceutically acceptable salt thereof, is the compound wherein subscript p is 1, 2, 3, or 4. In some embodiments, the compound or pharmaceutically acceptable salt thereof, is the compound wherein subscript p is 1 or 2. In some embodiments, the compound or pharmaceutically acceptable salt thereof, is the compound wherein subscript p is 1.

In some embodiments, the compound or pharmaceutically acceptable salt thereof, is the compound wherein Ring A is a 5 to 6 membered heterocycloalkyl having 1 heteroatom of N, O or S, or a 5 to 6 membered heteroaryl having 1 or 2 heteroatoms each independently N, O or S. In some embodiments, the compound or pharmaceutically acceptable salt thereof, is the compound wherein Ring A is a 5 to 6 membered heterocycloalkyl having 1 heteroatom of N or O, or a 5 to 6 membered heteroaryl having 1N heteroatom. In some embodiments, the compound or pharmaceutically acceptable salt thereof, is the compound wherein Ring A is pyrrolidinyl, piperidinyl, tetrahydropyranyl, or pyridyl. In some embodiments, the compound or pharmaceutically acceptable salt thereof, is the compound wherein Ring A is tetrahydropyranyl.

In some embodiments, the compound or pharmaceutically acceptable salt thereof, is the compound wherein each R¹ is independently C₁₋₆ alkyl. In some embodiments, the compound or pharmaceutically acceptable salt thereof, is the compound wherein each R¹ is independently C₁₋₃ alkyl. In some embodiments, the compound or pharmaceutically acceptable salt thereof, is the compound wherein each R¹ is methyl, ethyl, n-propyl or iso-propyl. In some embodiments, the compound or pharmaceutically acceptable salt thereof, is the compound wherein each R¹ is Me.

In some embodiments, the compound or pharmaceutically acceptable salt thereof, is the compound wherein R³ is hydrogen or halogen. In some embodiments, the compound or pharmaceutically acceptable salt thereof, is the compound wherein R³ is halogen. In some embodiments, the compound or pharmaceutically acceptable salt thereof, is the compound wherein R³ is F or Cl. In some embodiments, the compound or pharmaceutically acceptable salt thereof, is the compound wherein R³ is hydrogen.

In some embodiments, the compound or pharmaceutically acceptable salt thereof, is the compound wherein each R⁴ is independently halogen or —CN. In some embodiments, the compound or pharmaceutically acceptable salt thereof, is the compound wherein each R⁴ is independently Cl or —CN. In some embodiments, the compound or pharmaceutically acceptable salt thereof, is the compound wherein each R⁴ is —CN.

In some embodiments, the compound or pharmaceutically acceptable salt thereof, is the compound having the structure of Formula Ia:

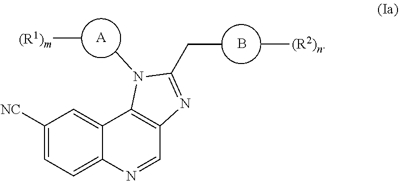

In some embodiments, the compound or pharmaceutically acceptable salt thereof, is the compound having the structure of Formula Ib:

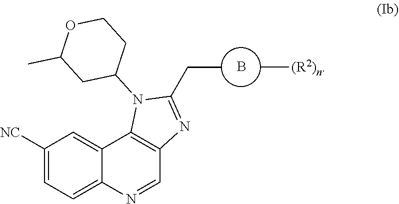

In some embodiments, the compound or pharmaceutically acceptable salt thereof, is the compound having the structure of Formula Ic:

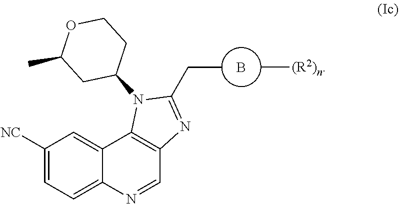

In some embodiments, the compound or pharmaceutically acceptable salt thereof, is the compound wherein Ring B is a 5 to 6 membered heteroaryl having 1 to 3 heteroatoms each independently N, O or S. In some embodiments, the compound or pharmaceutically acceptable salt thereof, is the compound wherein Ring B is a 5 to 6 membered heteroaryl having 2 to 3 heteroatoms each independently N, O or S. In some embodiments, the compound or pharmaceutically acceptable salt thereof, is the compound wherein Ring B is pyrrolyl, pyrazolyl, imidazolyl, isoxazolyl, oxazolyl, isothiazolyl, thiazolyl, triazolyl, oxadiazolyl, pyridyl, pyridazinyl, pyrimidinyl, or pyrazinyl. In some embodiments, the compound or pharmaceutically acceptable salt thereof, is the compound wherein Ring B is pyrazolyl, isoxazolyl, 1,2,3-triazolyl, 1,2,4-oxadiazolyl, 1,3,4-oxadiazolyl, or pyridyl.

In some embodiments, the compound or pharmaceutically acceptable salt thereof, is the compound wherein the group

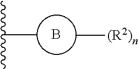

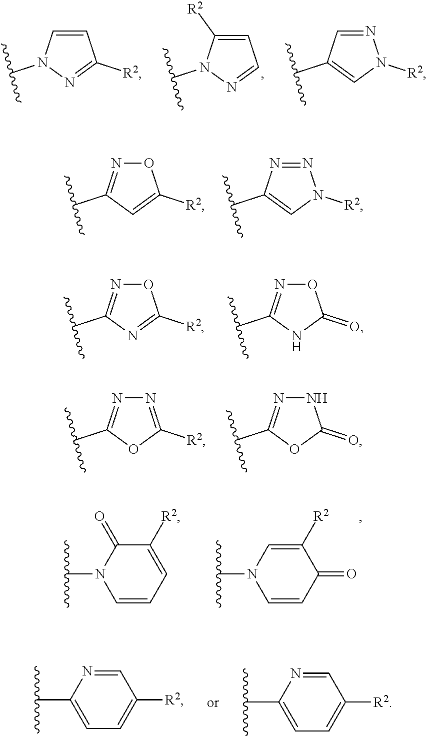

In some embodiments, the compound or pharmaceutically acceptable salt thereof, is the compound wherein each R² is C₁₋₃ alkyl, ═O, C(O)OR^{2b}, OC(O)R^{2a}, S(O)₂R^{2a}, N(R^{2b})S(O)₂R^{2a}, S(O)₂N(R^{2b})(R^{2c}), C₃₋₆ cycloalkyl, or 3 to 6 membered heterocycloalkyl having 1 to 3 heteroatoms each independently N, O or S, wherein each alkyl or cycloalkyl is substituted with 1 to 2 R^{2d} groups, and wherein each heterocycloalkyl is optionally substituted with 1 to 2 R^{2e} groups;
each R^{2b} and R^{2c} is hydrogen or C₁₋₃ alkyl;
each R^{2d} is independently C(O)R^{2d1} or S(O)₂R^{2d1};
each R^{2e} is independently C₁₋₃ alkyl, —OH, ═O, C(O)R^{2e1} or S(O)₂R^{2e1}; and each R^{2a}, R^{2d1} and R^{2e1} is independently C₁₋₃ alkyl.

In some embodiments, the compound or pharmaceutically acceptable salt thereof, is the compound wherein each R² is independently ═O, —COOH, —C(O)OMe, —SO₂Me, —NHSO₂Me, —CH₂CH₂SO₂Me,

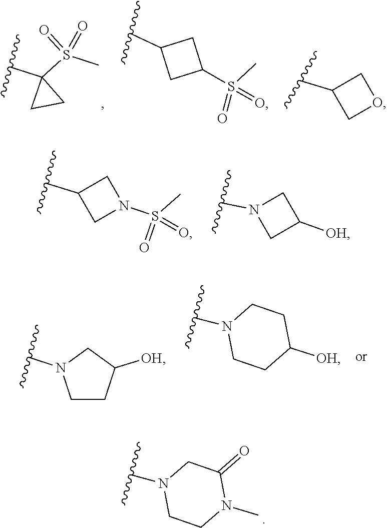

In some embodiments, the compound or pharmaceutically acceptable salt thereof, is the compound wherein the group

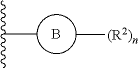

is

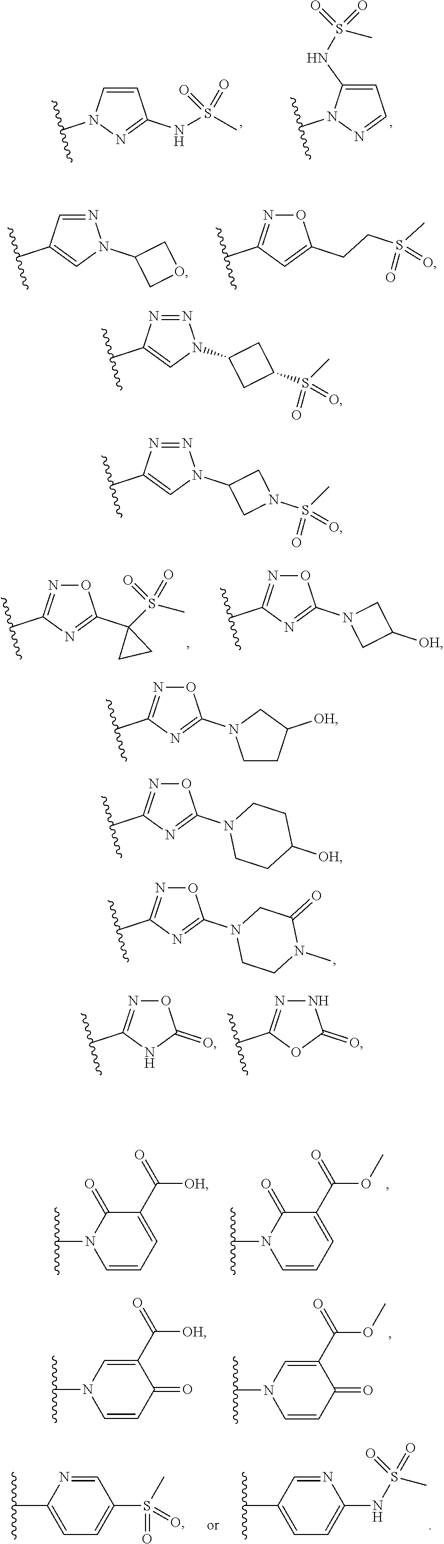

In some embodiments, the compound or pharmaceutically acceptable salt thereof, is the compound having the structure of a compound in Table 1.

TABLE 1
Compounds

| Com- | |
|---|---|
| pound | Structure |
| 1 | 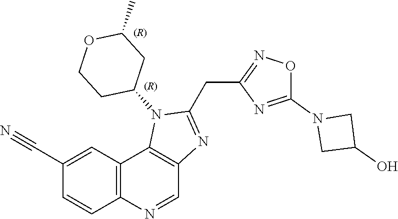 |
| 2 | 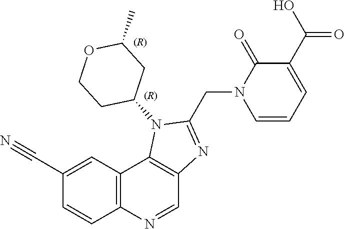 |
| 3 | 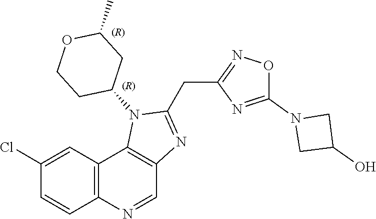 |
| 4 | 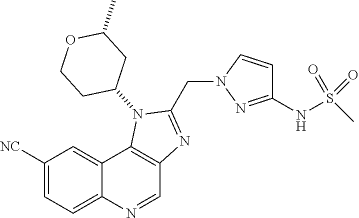 |
| 5 | 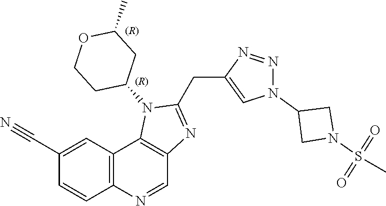 |
| 6 | 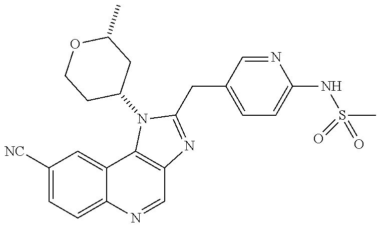 |
| 7 | 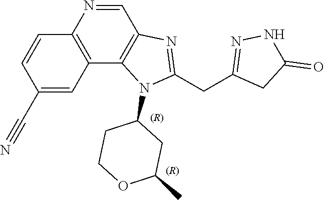 |
| 8 | 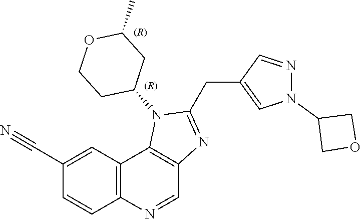 |
| 9 | 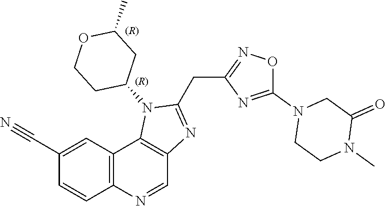 |
| 10 | 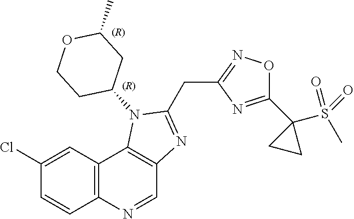 |
| 11 | 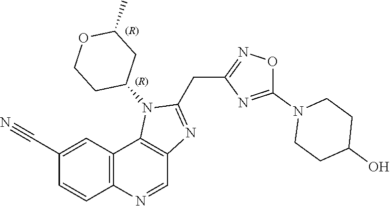 |
| 12 | 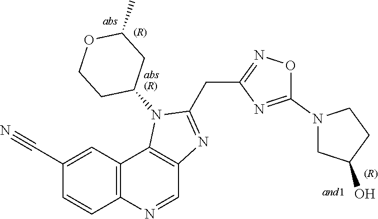 |
| 13 | 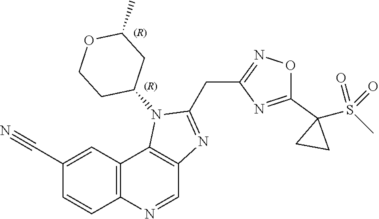 |
| 14 | 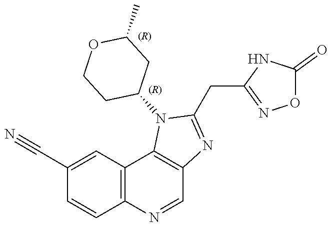 |
| 15 | 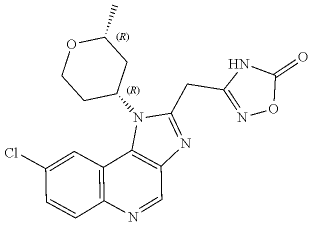 |
| 16 | 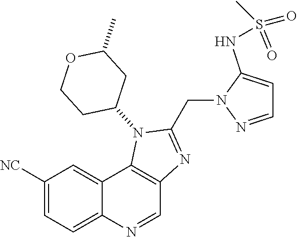 |
| 17 | 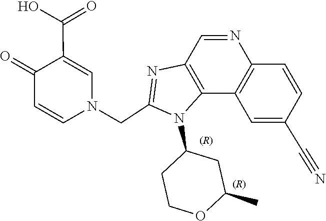 |
| 18 | 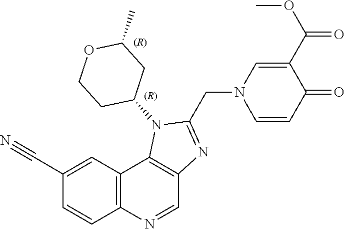 |
| 19 | 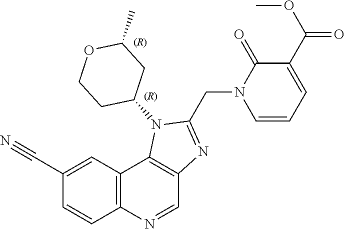 |
| 20 | 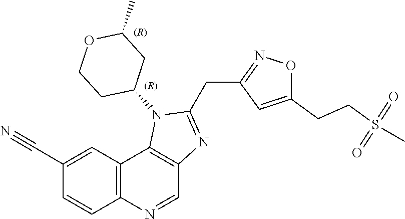 |
| 21 | 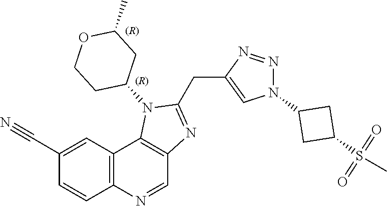 |
| 22 | 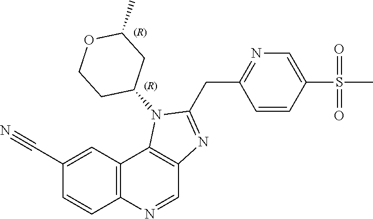 |

The compounds of the present disclosure as described herein may be prepared and/or formulated as pharmaceutically acceptable salts or when appropriate as a free base. Pharmaceutically acceptable salts are non-toxic salts of a free base form of a compound that possesses the desired pharmacological activity of the free base. These salts may be derived from inorganic or organic acids or bases. Examples of a pharmaceutically acceptable salt of the compound of Formula (I) of the present disclosure include an inorganic acid salt such as hydrochloride, sulfate, carbonate, and phosphate etc., and an organic acid salt such as fumarate, maleate, methanesulfonate, and p-toluenesulfonate etc. Further salts with an alkaline metal such as sodium, potassium etc., with an alkaline earth metal such as magnesium or calcium etc., with an organic amine such as a lower alkyl amine, or a lower alcoholamine, with a basic amino acid such as lysine, arginine, ornithine, or an ammonium salt is also included. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington: The Science and Practice of Pharmacy, 21^{st} Edition, Lippincott Williams and Wilkins, Philadelphia, Pa., 2006.

In some embodiments, the compound of the present disclosure described herein or pharmaceutically acceptable salt, isomer, or a mixture thereof, is a compound in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula (I) can generally be prepared by conventional techniques known to those skilled in the art or by processes analogous to those described in the Examples as set out below using an appropriate isotopically-labeled reagent in place of the non-labeled reagent previously employed.

The compounds of the embodiments disclosed herein, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

Activity against LRRK2 can be measured by any biochemical assay known in the art to be useful for evaluating LRRK2, for example, those commercially available, such as LRRK2 ELISA kit (Aviva Systems, San Diego, CA USA) and LRRK2 kinase enzyme system (Promega Corp.), those described in U.S. Pat. Nos. 10,039,753 and 11,161,844, as well as an assay described herein. In some embodiments, the compound of the present disclosure comprises an activity against LRRK2, wherein the IC₅₀ is less than about 30 μM, such as less than about 20 μM, less than about 10 μM, less than about 1 μM, less than about 0.1 μM, less than about 0.01 μM, less than about 0.001 μM, or less than about 0.0001 μM, in a biochemical assay.

Activity against LRRK2 can also be measured by any cell assay known in the art to be useful for evaluating LRRK2, for example, the phospho-LRRK2 (Ser935) cellular kit (Cisbio Bioassays, France), as described in Hermanson, S B et al. PLOS ONE 7(8): e43580, and as described herein. In some embodiments, the compound of the present disclosure comprises an activity against LRRK2, wherein the IC₅₀ is less than about 30 μM, such as less than about 20 μM, less than about 10 μM, less than about 1 μM, less than about 0.1 μM, less than about 0.01 μM, less than about 0.001 μM, or less than about 0.0001 μM, in a cellular assay.

In some embodiments, a compound of the present disclosure has selectivity for LRRK2 over one or more of the other kinases, for example, LRRK1, LIMK1, LIMK2, RIPK1, RIPK2, RIPK3, ANKRD3, SgK288, IRAK1, IRAK2, IRAK3, IRAK4, JAK1, JAK2, JAK3, TESK1, and/or TESK2. Selectivity can be measured by relative values in corresponding biochemical assays, e.g., activity to inhibit LRRK2 over LRRK1, LIMK1, LIMK2, RIPK1, RIPK2, RIPK3, ANKRD3, SgK288, IRAK1, IRAK2, IRAK3, IRAK4, JAK1, JAK2, JAK3, TESK1, and/or TESK2.

In some embodiments, a compound of the present disclosure has selectivity for LRRK2 of at least about 1.2, about 1.5, about 2, about 3, about 4, about 5, about 6, about 7, about 8, about 9, about 10, about 15, about 20, about 30, about 40, about 50, about 60, about 70, about 80, about 90, about 100, about 200, about 300, about 400, about 500, about 1000, about 2000, about 3000, about 4000, about 5000, or about 10000-fold or more over one or more, for example, 2, 3, 4, 5, 6, 7, 8, or 9 or more, other kinases including LRRK1, LIMK1, LIMK2, RIPK1, RIPK2, RIPK3, ANKRD3, SgK288, IRAK1, IRAK2, IRAK3, IRAK4, JAK1, JAK2, JAK3, TESK1, and/or TESK2.

### IV. Pharmaceutical Compositions

In some embodiments, a pharmaceutical composition comprises a pharmaceutically effective amount of a compound of the present disclosure, or a pharmaceutically acceptable salt thereof, and a pharmaceutically acceptable carrier or excipient. In some embodiments, a pharmaceutical composition comprises a pharmaceutically effective amount of a compound of Formula (I), (Ia), (Ib), or (Ic), or a pharmaceutically acceptable salt, and/or solvate thereof, and a pharmaceutically acceptable carrier or excipient.

In some embodiments, the pharmaceutical composition further comprises one or more additional therapeutic agents. Any suitable additional therapeutic agent or combination therapy can be used with the compounds of Formula (I), (Ia), (Ib), or (Ic), or a pharmaceutically acceptable salt thereof, such as the agents and therapies described within.

In some embodiments, the pharmaceutical composition comprises a compound of Formula (I), (Ia), (Ib), or (Ic), and an additional therapeutic agent, wherein the additional therapeutic agent is an anti-Parkinson's disease agent.

In some embodiments, the pharmaceutical composition comprises a compound of Formula (I), (Ia), (Ib), or (Ic), and an additional therapeutic agent, wherein the additional therapeutic agent is an anti-inflammatory bowel disease agent.

The compounds herein are formulated with conventional carriers and excipients. Tablets will contain excipients, glidants, fillers, binders and the like. Aqueous formulations are prepared in sterile form, and when intended for delivery by other than oral administration generally will be isotonic. All formulations will optionally contain excipients such as those set forth in the “Handbook of Pharmaceutical Excipients” (1986). Excipients include ascorbic acid and other antioxidants, chelating agents such as EDTA, carbohydrates such as dextran, hydroxyalkylcellulose, hydroxyalkylmethylcellulose, stearic acid and the like. The pH of the formulations ranges from about 3 to about 11, for example about 7 to 10.

While it is possible for the active ingredients to be administered alone it may be preferable to present them as pharmaceutical formulations. The formulations, both for veterinary and for human use, comprise at least one active ingredient, as above defined, together with one or more acceptable carriers and optionally other therapeutic ingredients, particularly those additional therapeutic ingredients as discussed herein. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any suitable method. Techniques and formulations generally are found in Remington's Pharmaceutical Sciences (Mack Publishing Co., Easton, PA). Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Pharmaceutical formulations herein comprise a combination together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, solutions, syrups or elixirs may be prepared. Compositions intended for oral use may be prepared according to any method for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions may be in the form of a sterile injectable or intravenous preparations, such as a sterile injectable aqueous or oleaginous suspension. This suspension may be formulated according to the known art using those suitable dispersing or wetting agents and suspending agents which have been mentioned above. The sterile injectable or intravenous preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10%, and particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns, such as 0.5, 1, 30, 35 etc., which is administered by rapid inhalation through the nasal passage or by inhalation through the mouth so as to reach the alveolar sacs. Suitable formulations include aqueous or oily solutions of the active ingredient.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations may include other agents conventional in the art having regard to the type of formulation in question, for example those suitable for oral administration may include flavoring agents.

In some embodiments, a veterinary composition comprises at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds herein are used to provide controlled release pharmaceutical formulations containing as active ingredient one or more of the compounds (“controlled release formulations”) in which the release of the active ingredient is controlled and regulated to allow less frequency dosing or to improve the pharmacokinetic or toxicity profile of a given active ingredient.

Effective dose of active ingredient depends at least on the nature of the condition being treated, toxicity, whether the compound is being used prophylactically (lower doses) or against an active viral infection, the method of delivery, and the pharmaceutical formulation, and will be determined by the clinician using conventional dose escalation studies. It can be expected to be from about 0.0001 to about 100 mg/kg body weight per day; typically, from about 0.01 to about 10 mg/kg body weight per day; more typically, from about 0.01 to about 5 mg/kg body weight per day; most typically, from about 0.05 to about 0.5 mg/kg body weight per day. For example, the daily candidate dose for an adult human of approximately 70 kg body weight will range from 1 mg to 1000 mg, preferably between 5 mg and 500 mg, and may take the form of single or multiple doses.

### V. Routes of Administration

One or more of the compounds of Formula (I), (Ia), (Ib), or (Ic) are administered by any route appropriate to the condition to be treated. Suitable routes include oral, rectal, nasal, pulmonary, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. An advantage of the compounds herein is that they are orally bioavailable and can be dosed orally.

The compounds of the present disclosure can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the preferred route may vary with for example the condition of the recipient. An advantage of certain compounds disclosed herein is that they are orally bioavailable and can be dosed orally.

A compound of the present disclosure may be administered to an individual in accordance with an effective dosing regimen for a desired period of time or duration, such as at least about one month, at least about 2 months, at least about 3 months, at least about 6 months, or at least about 12 months or longer. In one variation, the compound is administered on a daily or intermittent schedule for the duration of the individual's life.

The dosage or dosing frequency of a compound of the present disclosure may be adjusted over the course of the treatment, based on the judgment of the administering physician.

The compound may be administered to an individual (e.g., a human) in an effective amount. In some embodiments, the compound is administered once daily.

The compound can be administered by any useful route and means, such as by oral or parenteral (e.g., intravenous) administration. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day, or such as from about 0.3 mg to about 30 mg per day, or such as from about 30 mg to about 300 mg per day.

A compound of the present disclosure may be combined with one or more additional therapeutic agents in any dosage amount of the compound of the present disclosure (e.g., from 1 mg to 1000 mg of compound). Therapeutically effective amounts may include from about 1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose. Other therapeutically effective amounts of the compound of the present disclosure are about 100, 125, 150, 175, 200, 225, 250, 275, 300, 325, 350, 375, 400, 425, 450, 475, or about 500 mg per dose. Other therapeutically effective amounts of the compound of the present disclosure are about 100 mg per dose, or about 125, 150, 175, 200, 225, 250, 275, 300, 350, 400, 450, or about 500 mg per dose. A single dose can be administered hourly, daily, or weekly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In some embodiments, a single dose can be administered once every week. A single dose can also be administered once every month.

Other therapeutically effective amounts of the compound of the present disclosure are about 20, 25, 30, 35, 40, 45, 50, 55, 60, 65, 70, 75, 80, 85, 90, 95, or about 100 mg per dose.

The frequency of dosage of the compound of the present disclosure are will be determined by the needs of the individual patient and can be, for example, once per day or twice, or more times, per day. Administration of the compound continues for as long as necessary to treat the viral infection. For example, a compound can be administered to a human being infected with a virus for a period of from 20 days to 180 days or, for example, for a period of from 20 days to 90 days or, for example, for a period of from 30 days to 60 days.

Administration can be intermittent, with a period of several or more days during which a patient receives a daily dose of the compound of the present disclosure followed by a period of several or more days during which a patient does not receive a daily dose of the compound. For example, a patient can receive a dose of the compound every other day, or three times per week. Again by way of example, a patient can receive a dose of the compound each day for a period of from 1 to 14 days, followed by a period of 7 to 21 days during which the patient does not receive a dose of the compound, followed by a subsequent period (e.g., from 1 to 14 days) during which the patient again receives a daily dose of the compound. Alternating periods of administration of the compound, followed by non-administration of the compound, can be repeated as clinically required to treat the patient.

In some embodiments, a pharmaceutical composition comprises a compound of the present disclosure, or a pharmaceutically acceptable salt thereof, in combination with one or more (e.g., one, two, three, four, one or two, one to three, or one to four) additional therapeutic agents, and a pharmaceutically acceptable excipient are provided.

### VI. Methods or Uses

In some embodiments, a method or use for inhibiting LRRK2 in a cell in need thereof, comprises administering to the cell an effective amount of a compound of the present disclosure, or a pharmaceutically acceptable salt thereof, or a pharmaceutical composition of the present disclosure. In some embodiments, the method or use of inhibiting LRRK2 in a cell comprises contacting the cell with an effective amount of a compound of Formula (I), (Ia), (Ib), or (Ic), or a pharmaceutically acceptable salt thereof, or a pharmaceutical composition comprising the compound or salt thereof.

In some embodiments, a method of inhibiting LRRK2 in a cell comprises contacting the cell with an effective amount of a compound of the disclosure, or pharmaceutically acceptable salt thereof.

Inhibition of LRRK2 enzyme activity can be measured by any assay method known in the art, such as an in vitro assay described in WO 2011/141756, WO 2012/028629, WO 2012/058193, WO 2017/046675, WO 2018/163030, WO 2018/163066, WO 2021/080929, or US 20210002260. Other illustrative in vitro assays can be found in the Examples herein. In some embodiments, an in vitro assay comprises an enzyme assay or a cell assay.

In some embodiments, the inhibition of LRRK2 enzyme activity is measured in an in vivo model. Illustrative in vivo models for LRRK2-associated diseases are described in Xiong, Y. et al. Adv Neurobiol. 2017; 14:163-191.

In some embodiments, a method of inhibiting LRRK2 comprises administering an effective amount of a compound of the present disclosure, thereby reducing LRRK2 activity in an assay described herein as compared to a control without administering the compound of the disclosure. In some embodiments, the LRRK2 activity is reduced by from about 5% to about 100%, such as from about 10% to about 97%, from about 20% to about 95%, from about 20% to about 90%, from about 20% to about 80%, or from about 20% to about 70%. In some embodiments, the LRRK2 activity is reduced by about 10%, about 20%, about 30%, about 40%, about 50%, about 60%, about 70%, about 80%, about 90%, or about 100%.

In some embodiments, a method or use for inhibiting LRRK2 in a subject in need thereof, comprises administering to the subject a therapeutically effective amount of a compound of the present disclosure, or a pharmaceutically acceptable salt thereof, or a pharmaceutical composition of the present disclosure. In some embodiments, a method or use of inhibiting LRRK2 in a subject in need thereof, comprises administering to the subject a therapeutically effective amount of a compound of Formula (I), (Ia), (Ib), or (Ic), or a pharmaceutically acceptable salt thereof, or a pharmaceutical composition comprising the compound or salt thereof.

In some embodiments, a method or use for treating a LRRK2-associated disease or condition, such as neurological disorders (for example, Parkinson's disease), and certain immunological disorders (such as inflammatory bowel disease like ulcerative colitis or Crohn's disease) comprises administering a therapeutically effective amount of a compound of Formula I or a pharmaceutically acceptable salt thereof, and a pharmaceutically acceptable excipient, to a subject in need thereof.

In some embodiments, a method of treating a LRRK2-associated disease or condition in a subject in need thereof comprises administering to the subject a therapeutically effective amount of a compound of the disclosure, or pharmaceutically acceptable salt thereof.

In some embodiments, the present disclosure provides a method or use for treating a LRRK2-associated disease or condition. In some embodiments, the LRRK2-associated disease or condition includes Parkinson's disease; brain injury; stroke; cerebrovascular diseases (including cerebral arteriosclerosis, cerebral amyloid angiopathy, hereditary cerebral hemorrhage, and brain hypoxia-ischemia); cognitive disorders (including amnesia, senile dementia, HIV-associated dementia, Alzheimer's disease, Huntington's disease, Lewy body dementia, vascular dementia, drug-related dementia, tardive dyskinesia, myoclonus, dystonia, delirium, Pick's disease, Creutzfeldt-Jacob disease, HIV disease, Gilles de la Tourette's syndrome, epilepsy, muscular spasms and disorders associated with muscular spasticity or weakness including tremors, and mild cognitive impairment); mental deficiency (including spasticity, Down syndrome and fragile X syndrome); sleep disorders (including hypersomnia, circadian rhythm sleep disorder, insomnia, parasomnia, and sleep deprivation) and psychiatric disorders such as anxiety (including acute stress disorder, generalized anxiety disorder, social anxiety disorder, panic disorder, post-traumatic stress disorder, agoraphobia, and obsessive-compulsive disorder); factitious disorder (including acute hallucinatory mania); impulse control disorders (including compulsive gambling and intermittent explosive disorder); mood disorders (including bipolar I disorder, bipolar II disorder, mania, mixed affective state, major depression, chronic depression, seasonal depression, psychotic depression, premenstrual syndrome (PMS), premenstrual dysphoric disorder (PDD), and postpartum depression); psychomotor disorder; psychotic disorders (including schizophrenia, schizoaffective disorder, schizophreniform, and delusional disorder); drug dependence (including narcotic dependence, alcoholism, amphetamine dependence, cocaine addiction, nicotine dependence, and drug withdrawal syndrome); eating disorders (including anorexia, bulimia, binge eating disorder, hyperphagia, obesity, compulsive eating disorders and pagophagia); sexual dysfunction disorders; urinary incontinence; neuronal damage disorders (including ocular damage, retinopathy or macular degeneration of the eye, tinnitus, hearing impairment and loss, and brain edema) and pediatric psychiatric disorders (including attention deficit disorder, attention deficit/hyperactive disorder, conduct disorder, and autism).

In some embodiments, the LRRK2-associated disease or condition is Parkinson's disease, Lewy body dementia, frontotemporal dementia, corticobasal dementia, progressive supranuclear palsy, Alzheimer's disease, tauopathy disease, or alpha-synucleinopathy. In some embodiments, the LRRK2-associated disease or condition is Parkinson's disease. In some embodiments, the LRRK2-associated disease or condition is frontotemporal dementia. In some embodiments, the LRRK2-associated disease or condition is corticobasal dementia. In some embodiments, the LRRK2-associated disease or condition is progressive supranuclear palsy. In some embodiments, the LRRK2-associated disease or condition is Alzheimer's disease. In some embodiments, the LRRK2-associated disease or condition is tauopathy disease. In some embodiments, the LRRK2-associated disease or condition is alpha-synucleinopathy.

In some embodiments, the LRRK2-associated disease or condition is an inflammatory bowel disease. In some embodiments, the inflammatory bowel disease is ulcerative colitis or Crohn's disease. In some embodiments, the inflammatory bowel disease is ulcerative colitis. In some embodiments, the inflammatory bowel disease is Crohn's disease.

Increased LRRK2 level and/or activity has been associated with an aberrant level of autophagy in certain cell types of Parkinson's disease patients. For instance, LRRK2 G2019S and LRRK2 R1441C mutations were associated with an increase in kinase activity and a decrease in autophagic flux via blocked clearance of autophagosomes. See, Madureira, M. et al. Frontiers in Neuroscience 2020, 14, Article 498, pages 1-19. Inhibition of LRRK2 G2019S kinase activity in cellular models enhanced autolysosome formation. See, Obergasteiger, et al. Cell Death Discovery 2020, 6 (45), pages 1-13.

A number of diseases are associated with aberrant levels of autophagy, and specifically, a decreased level of autophagy compared to a healthy subject. See, Ichimiya, et al. Intl. J. Mol. Sci. 2020, 21, 8974, pages 1-21. Any of autophagy-related diseases or conditions may benefit from LRRK2 inhibition through administration of the compound of the present disclosure or pharmaceutically acceptable salt thereof.

Accordingly, in some embodiments, the LRRK2-associated disease or condition is an autophagy-related disease or condition. In some embodiments, the autophagy-related disease or condition relates to decreased levels of one or more of mitophagy, allophagy, ER-phagy, lysophagy, nucleophagy, pexophagy, lipophagy, xenophagy, aggrephagy, ribophagy, NPC-phagy, and RN/RN-autophagy as compared to a level in a control subject. In some embodiments, the autophagy-related disease or condition is a liver disease (for example, non-alcoholic fatty liver disease (NAFLD), alpha 1-antitrypsin deficiency (AATD), or hereditary hypofibrinogenemia with hepatic storage (HHHS)), a kidney disease (for example, type 1 diabetes mellitus, type 2 diabetes mellitus, acute renal injury, and chronic kidney disease caused by diabetes mellitus, hypertension or chronic nephritis), a heart disease (for example, heart failure), an inflammatory bowel disease (for example, Crohn's disease), or a neurodegenerative disease (for example, Parkinson's disease). In some embodiments, the autophagy-related disease or condition is alpha 1-antitrypsin deficiency (AATD).

In some embodiments, a use of the present invention for the manufacture of a medicament for treating a LRRK2-associated disease or condition comprises a compound or a pharmaceutical composition as described herein.

In some embodiments, a compound or composition for use of the present invention for treating a LRRK2-associated disease or condition comprises a compound or a pharmaceutical composition as described herein.

In some embodiments, a kit is suitable for use in performing a method or use described above. In some embodiments, the kit of the present invention comprises one or more compounds of the invention. In some embodiments, the kit comprises a first dosage form comprising one or more of the compounds of the present invention and a container for the dosage, in quantities sufficient to carry out the methods or uses of the present invention.

### VII. EXAMPLES

Many general references providing commonly known chemical synthetic schemes and conditions useful for synthesizing the disclosed compounds are available (see, e.g., Smith, March's Advanced Organic Chemistry: Reactions, Mechanisms, and Structure, 7^{th} edition, Wiley-Interscience, 2013.)

Compounds as described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. For example, disclosed compounds can be purified via silica gel chromatography. See, e.g., Introduction to Modern Liquid Chromatography, 2nd ed., ed. L. R. Snyder and J. J. Kirkland, John Wiley and Sons, 1979; and Thin Layer Chromatography, E. Stahl (ed.), Springer-Verlag, New York, 1969.

Compounds were characterized using standard instrumentation methods. Identification of the compound was carried out by hydrogen nuclear magnetic resonance spectrum (¹H-NMR) and mass spectrum (MS). ¹H-NMR was measured at 400 MHz, unless otherwise specified. In some cases, exchangeable hydrogen could not be clearly observed depending on the compound and measurement conditions. The designation br. or broad, used herein, refers to a broad signal. HPLC preparative chromatography was carried out by a commercially available ODS column in a gradient mode using water/methanol (containing formic acid) as eluents, unless otherwise specified.

Certain abbreviations and acronyms are used in describing the experimental details. Although most of these would be understood by one skilled in the art, the Table below contains a list of many of these abbreviations and acronyms.

TABLE 2
List of abbreviations and acronyms.

| Abbreviation | Meaning |
|---|---|
| Ac | acetate |
| ACN | acetonitrile |
| DCM | dichloromethane |
| DIPEA or DIEA | diisopropylethylamine |
| DMB | 2, 4-dimethoxybenzyl |
| DMSO | dimethylsulfoxide |
| DMF | dimethylformamide |
| Et | ethyl |
| EtOAc | ethylacetate |
| HPLC | high performance liquid chromatography |
| LC | liquid chromatography |
| LCMS | liquid chromatography-mass spectrometry |
| Me | methyl |
| MS or ms | mass spectrum |
| MTBE | methyl tert-butyl ether |
| NBS | N-bromosuccinimide |
| NMR | nuclear magnetic resonance |
| rpm | revolutions per minute |
| T3P or T₃P | propanephosphonic acid anhydride |
| TBS | tert-butyldimethylsilyl |
| TBSCl | tert-butyldimethylsilyl chloride |
| tBuBrettPhos | [(2-di-tert-butylphosphino-3, 6-dimethoxy-2′,4′,6′-triisopropyl-1,1′- |
| Pd G3 | biphenyl)-2-(2′-amino-1,1′-biphenyl)]palladium(II) methanesulfonate |
| TEA | triethylamine |
| TFA | trifluoroacetic acid |
| THF | tetrahydrofuran |
| δ | parts per million referenced to residual non-deuterated solvent peak |

The Examples provided herein describe the synthesis of compounds disclosed herein as well as intermediates used to prepare the compounds. It is to be understood that individual steps described herein may be combined. It is also to be understood that separate batches of a compound may be combined and then carried forth in the next synthetic step.

In the following description of the Examples, specific embodiments are described. These embodiments are described in sufficient detail to enable those skilled in the art to practice certain embodiments of the present disclosure. Other embodiments may be utilized and logical and other changes may be made without departing from the scope of the disclosure. The following description is, therefore, not intended to limit the scope of the present disclosure.

Representative syntheses of compounds of the present disclosure are described in schemes below, and the particular examples that follow.

### Intermediate 1

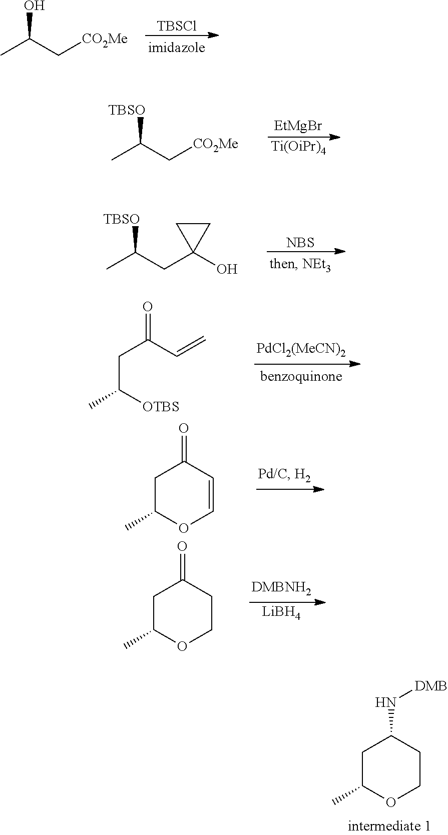

methyl (R)-3-((tert-butyldimethylsilyl)oxy)butanoate: To a solution of methyl (R)-3-hydroxybutanoate (19.4 mL, 169 mmol, 1.0 eq) in DCM (400 mL, 0.4 M) was added TBSCl (41.5 mL, 339 mmol, 2.0 eq) and imidazole (46 g, 677 mmol, 4.0 eq) at 0° C. The mixture was stirred at 20° C. for 12 hours under N₂ atmosphere. The mixture was diluted with water (100 mL), extracted with DCM (3×100 mL), dried over Na₂SO₄, filtered and concentrated under reduced pressure. The residue was purified by flash silica gel chromatography (petroleum ether/EtOAc=1/0 to 9/1) to give the title compound as a colorless oil. ¹H NMR (400 MHz, CDCl₃) δ ppm: 4.36-4.23 (m, 1H), 3.67 (s, 3H), 2.53-2.34 (m, 2H), 1.20 (d, J=6.0 Hz, 3H), 0.87 (s, 9H), 0.06 (d, J=9.2 Hz, 6H).

(R)-1-(2-((tert-butyldimethylsilyl)oxy)propyl)cyclopropan-1-ol: To a solution of methyl (R)-3-((tert-butyldimethylsilyl)oxy)butanoate (39 g, 151 mmol, 1.0 eq) in THF (350 mL, 0.4 M) was added tetraisopropoxytitanium (44 mL, 151 mmol, 1.0 eq) and EtMgBr (3 M in THF, 151 mL, 453 mmol, 3.0 eq) at 0° C. The mixture was stirred at 20° C. for 2 hours. The mixture was quenched with saturated NH₄Cl (200 mL) and 20% citric acid (200 mL) at 0° C. MTBE (200 mL) was added. The mixture was stirred at 0° C. for 20 min and filtered. The filtrate was extracted with MTBE (3×200 mL), dried over Na₂SO₄, filtered and the filtrate was concentrated under reduced pressure to give the title compound as a brown oil, which was used in next step without further purification. ¹H NMR (400 MHz, CDCl₃) δ ppm: 4.31-4.18 (m, 1H), 1.85-1.76 (m, 1H), 1.65-1.58 (m, 1H), 1.26 (d, J=6.4 Hz, 3H), 0.93-0.89 (m, 9H), 0.82-0.76 (m, 1H), 0.72-0.65 (m, 1H), 0.49-0.43 (m, 1H), 0.40-0.34 (m, 1H), 0.13 (s, 3H), 0.12 (s, 3H).

(R)-5-((tert-butyldimethylsilyl)oxy)hex-1-en-3-one: To a solution of (R)-1-(2-((tert-butyldimethylsilyl)oxy)propyl)cyclopropan-1-ol (35 g, 152 mmol, 1.0 eq) in DCM (400 mL, 0.4 M) was added NBS (27 g, 152 mmol, 1.0 eq). The mixture was stirred at 0° C. for 1 hour. TEA (42.3 mL, 304 mmol, 2.0 eq) was added, and the mixture was stirred at 0° C. for 2 hours. The mixture was diluted with 20% citric acid (200 mL) and the layers were separated. The organic layer was washed with saturated NaHCO₃ (3×200 mL), dried over Na₂SO₄, filtered and hydroquinone (200 mg) was added to the organic layer. The organic layer was concentrated under reduced pressure. The residue was filtered through a silica gel pad (300 g) and the filter cake was washed with DCM (800 mL). The filtrate and rinses were combined and concentrated under atmospheric pressure to give the title compound as a brown oil, which was used in the next step without further purification. ¹H NMR (400 MHz, CDCl₃) δ ppm: 6.46-6.30 (m, 1H), 6.29-6.17 (m, 1H), 5.96-5.79 (m, 1H), 4.39-4.28 (m, 1H), 2.85 (dd, J=7.2, 14.8 Hz, 1H), 2.54 (dd, J=5.2, 14.8 Hz, 1H), 1.19 (d, J=6.0 Hz, 3H), 0.86-0.82 (m, 9H), 0.05 (m, 3H), 0.01 (s, 3H).

(R)-2-methyl-2,3-dihydro-4H-pyran-4-one: A mixture of (R)-5-((tert-butyldimethylsilyl)oxy)hex-1-en-3-one (10 g, 39 mmol, 1.0 eq), PdCl₂(MeCN)₂ (102 mg, 394 mol, 0.01 eq), benzoquinone (8.9 mL, 39 mmol, 1.0 eq) and H₂O (3.4 mL, 189 mmol, 4.8 eq) in acetone (50 mL, 0.8 M) was stirred at 40° C. for 6 hours under N₂ atmosphere. The solvent was removed under reduced pressure, and the residue was dissolved in DCM (30 mL). The solution was filtered, and the filtrate was concentrated under reduced pressure to give a residue. The residue was distilled under vacuum (50° C., oil pump, 10 mmHg) to give the title compound as a colorless oil. ¹H NMR (400 MHz, CDCl₃) δ ppm: 7.33 (d, J=6.0 Hz, 1H), 5.39 (dd, J=1.2, 6.0 Hz, 1H), 4.61-4.48 (m, 1H), 2.62-2.34 (m, 2H), 1.45 (d, J=6.4 Hz, 3H). [α]²⁵D=+167.009 (c=0.109, CHCl3).

(R)-2-methyltetrahydro-4H-pyran-4-one: A mixture of (R)-2-methyl-2,3-dihydro-4H-pyran-4-one (1.0 g, 8.03 mmol, 1.0 eq) and Pd/C (500 mg, 10 wt %) in THF (20 mL, 0.4 M) was stirred at 20° C. for 1 hour under H₂ (15 psi). The mixture was filtered and concentrated under reduced pressure to give the title compound as a colorless oil, which was used in the next step without further purification. ¹H NMR (400 MHz, CDCl₃) δ ppm: 4.31-4.23 (m, 1H), 3.79-3.63 (m, 2H), 2.64-2.51 (m, 1H), 2.44-2.21 (m, 3H), 1.32 (d, J=6.4 Hz, 3H).

Intermediate 1: A mixture of (R)-2-methyltetrahydro-4H-pyran-4-one (200 mg, 1.75 mmol, 1.0 eq) and (2,4-dimethoxyphenyl)methanamine (396 μL, 2.63 mmol, 1.5 eq) in MeOH (5 mL, 0.4 M) was stirred at 20° C. for 1 hour. The mixture was cooled to −78° C. and LiBH₄ (38.2 mg, 1.75 mmol, 1.0 eq) was added. The mixture was stirred at 20° C. for 12 hours. Then the mixture was quenched with saturated Na₂CO₃ (20 mL) and diluted with water (20 mL). The mixture was extracted with DCM (3×20 mL), dried over Na₂SO₄, filtered and concentrated under reduced pressure. The resulting crude material was purified by flash silica gel chromatography (DCM/MeOH=1/0 to 9/1) to give the title compound as brown oil. [M+H]⁺=266.2. ¹H NMR (400 MHz, CDCl₃) δ ppm: 7.12 (d, J=8.0 Hz, 1H), 6.51-6.34 (m, 2H), 4.06-3.92 (m, 1H), 3.80 (s, 3H), 3.79 (s, 3H), 3.75 (s, 2H), 3.44-3.34 (m, 2H), 2.73-2.61 (m, 1H), 1.92-1.85 (m, 1H), 1.84-1.77 (m, 1H), 1.46-1.30 (m, 1H), 1.18 (d, J=6.0 Hz, 3H), 1.15-1.03 (m, 1H).

### Intermediate 2

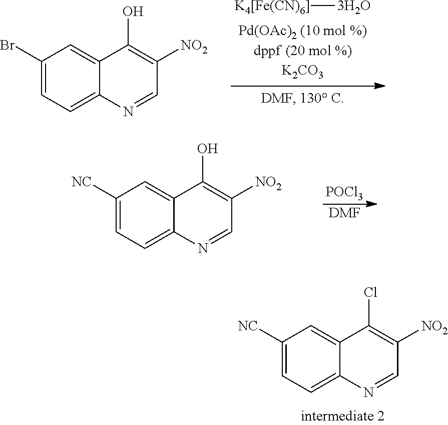

4-hydroxy-3-nitroquinoline-6-carbonitrile: DMF (60 mL, 0.3 M) was added to a flask charged with compound 11 (5 g, 18.6 mmol, 1 eq), tetrapotassium hexacyanoferrate(II) trihydrate (4.71 g, 11.15 mmol, 0.6 eq), dppf (2.06 g, 3.72 mmol, 0.2 eq), Pd(OAc)₂ (417.22 mg, 1.86 mmol, 0.1 eq) and K₂CO₃ (3.08 g, 22.3 mmol, 1.2 eq) under N₂ (g). The mixture was stirred at 130° C. for 12 hours, then the reaction mixture was cooled to room temperature and filtered through a celite pad. The filter cake was slowly rinsed with DMF (50 mL) and MTBE (600 mL) while the filtrate was stirred. A dark solid precipitated from the filtrate during the stirring. The resulting mixture was stirred at 20° C. for 15 minutes and then filtered. The second filtrate was concentrated in vacuo to a volume of approximately 10 mL, which was diluted with MTBE (30 mL), and the resulting dark precipitate was collected by filtration and triturated with ethyl acetate (30 mL) to give the title compound as a dark green solid, which was used in the next step without further purification. LCMS [M+H]⁺=215.9.

Intermediate 2: POCl₃ (2.18 mL, 23.4 mmol, 2.8 eq) was added to a solution of 4-hydroxy-3-nitroquinoline-6-carbonitrile (1.8 g, 8.37 mmol, 1.0 eq) in DMF (60 mL, 0.1 M) under N₂ (g). After stirring at 20° C. for 12 h, the mixture was quenched by adding water (30 mL) and the solution was extracted with EtOAc (3×30 mL). The combined organic phases were dried over Na₂SO₄, filtered and the filtrate was concentrated in vacuo. The residue was purified via silica gel chromatography (petroleum ether/EtOAc=1/0 to 85/15) to give the title compound as a white solid. ¹H NMR (400 MHz, CDCl₃) δ ppm: 9.39 (s, 1H), 8.84 (d, J=1.2 Hz, 1H), 8.36 (d, J=8.8 Hz, 1H), 8.10 (dd, J=1.6, 8.4 Hz, 1H).

### Intermediate 3

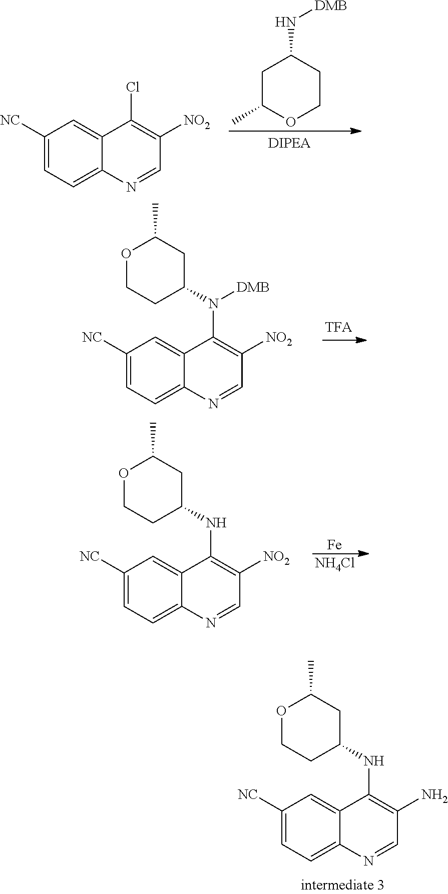

4-((3,4-dimethylbenzyl)((2R,4R)-2-methyltetrahydro-2H-pyran-4-yl)amino)-3-nitroquinoline-6-carbonitrile: DIPEA (64.3 μL, 369 μmol, 1.15 eq) was added to a solution of 4-chloro-3-nitroquinoline-6-carbonitrile (79.0 mg, 321 μmol, 1.0 eq) and (2R,4R)—N-(3,4-dimethylbenzyl)-2-methyltetrahydro-2H-pyran-4-amine (94.7 mg, 321 μmol, 1.0 eq) in MeCN (5 mL, 0.06 M) under N₂ (g). The mixture was stirred at 20° C. for 2 h. The solvent was removed in vacuo and the residue was dissolved with EtOAc (30 mL). The solution was washed with brine (3×10 mL), dried over Na₂SO₄, filtered and the filtrate was concentrated in vacuo. The residue was purified via silica gel chromatography (petroleum ether/EtOAc=1/0 to 4/1) to yield the title compound as an orange oil. LCMS [M+H]⁺=463.2. ¹H NMR (400 MHz, DMSO-d₆) δ ppm: 9.19 (s, 1H), 8.55 (s, 1H), 8.16 (d, J=0.8 Hz, 2H), 6.88 (d, J=8.0 Hz, 1H), 6.31-6.24 (m, 2H), 4.39-4.27 (m, 2H), 3.92-3.77 (m, 2H), 3.63 (s, 3H), 3.42 (s, 3H), 3.41-3.35 (m, 2H), 1.97-1.85 (m, 2H), 1.83-1.71 (m, 1H), 1.48 (q, J=11.6 Hz, 1H), 1.09 (d, J=6.2 Hz, 3H).

4-(((2R,4R)-2-methyltetrahydro-2H-pyran-4-yl)amino)-3-nitroquinoline-6-carbonitrile: TFA (43.2 μL, 583 μmol, 3.0 eq) was added to a solution of 4-((3,4-dimethylbenzyl)((2R,4R)-2-methyltetrahydro-2H-pyran-4-yl)amino)-3-nitroquinoline-6-carbonitrile (94.7 mg, 195 μmol, 1.0 eq) in DCM (2 mL, 0.1 M) under N₂ (g). The mixture was stirred at 20° C. for 2 h. The mixture was concentrated to a volume of 5 mL and treated with saturated aqueous sodium bicarbonate solution (20 mL). The aqueous layer was extracted with DCM (3×20 mL), and the combined organic layers were dried over Na₂SO₄, filtered, and concentrated in vacuo to give the title compound as a yellow solid, which was used in the next step without further purification. LCMS [M+H]⁺=313.2. ¹H NMR (400 MHz, DMSO-d₆) δ ppm: 9.08 (s, 2H), 8.29 (d, J=8.8 Hz, 1H), 8.15 (d, J=8.8 Hz, 1H), 8.01 (d, J=8.8 Hz, 1H), 3.91 (dd, J=4.0, 11.2 Hz, 1H), 3.80-3.75 (m, 1H), 3.44-3.37 (m, 2H), 2.04-1.97 (m, 2H), 1.95-1.88 (m, 1H), 1.73-1.64 (m, 1H), 1.12 (d, J=6.0 Hz, 3H).

Intermediate 3: H₂O (0.5 mL) and EtOH (2 mL) were added to a mixture of 4-(((2R,4R)-2-methyltetrahydro-2H-pyran-4-yl)amino)-3-nitroquinoline-6-carbonitrile (70 mg, 224 μmol, 1.0 eq), NH₄Cl (120 mg, 2.24 mmol, 10 eq) and Fe (125 mg, 2.24 mmol, 10 eq). The reaction mixture was heated to 80° C. for 1 hour. Then the mixture was diluted with ethanol (20 mL) and filtered. The filtrate was concentrated in vacuo, and the resulting solid was partitioned between saturated aqueous sodium bicarbonate solution (20 mL) and DCM (30 mL). The organic layer was dried over Na₂SO₄, filtered, and concentrated under reduced pressure to afford the title compound as a brown solid, which was used directly in the next step without further purification. LCMS [M+H]⁺=283.2. ¹H NMR (400 MHz, MeOD-d₄) δ ppm: 8.55 (d, J=1.6 Hz, 1H), 8.51 (s, 1H), 7.90 (d, J=8.4 Hz, 1H), 7.60 (dd, J=1.6, 8.8 Hz, 1H), 3.99-3.94 (m, 1H), 3.65-3.58 (m, 1H), 3.48-3.40 (m, 2H), 1.93-1.79 (m, 2H), 1.67-1.58 (m, 1H), 1.30-1.28 (m, 1H), 1.17 (d, J=6.4 Hz, 3H).

### Intermediate 4

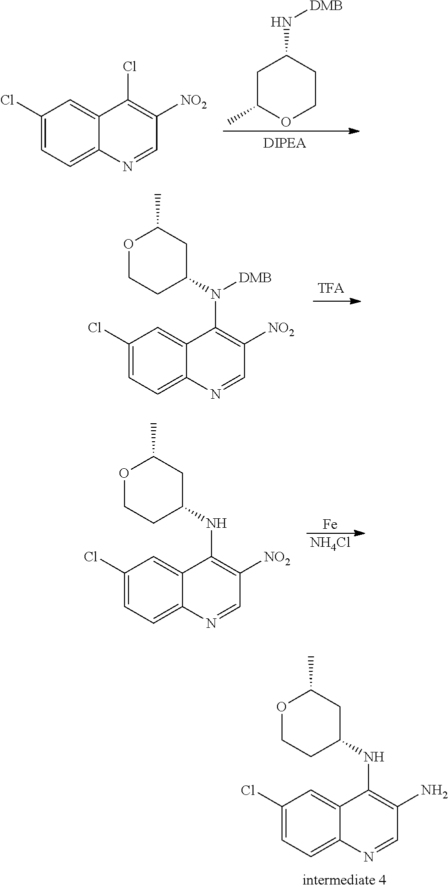

Intermediate 4 was prepared via the same method as intermediate 3 using 4,6-dichloro-3-nitroquinoline as the starting material.

6-chloro-N-(3,4-dimethylbenzyl)-N-((2R,4R)-2-methyltetrahydro-2H-pyran-4-yl)-3-nitroquinolin-4-amine: LCMS [M+H]⁺=472.2. ¹H NMR (400 MHz, DMSO-d₆) δ ppm: 9.06 (s, 1H), 8.15 (d, J=2.4 Hz, 1H), 8.03 (d, J=8.8 Hz, 1H), 7.88 (dd, J=2.4, 8.9 Hz, 1H), 6.82 (d, J=8.4 Hz, 1H), 6.30-6.23 (m, 2H), 4.27 (br s, 2H), 3.93-3.83 (m, 1H), 3.80-3.70 (m, 1H), 3.62 (s, 3H), 3.46 (s, 3H), 3.41-3.34 (m, 2H), 1.94 (d, J=13.6 Hz, 1H), 1.89-1.81 (m, 1H), 1.79-1.64 (m, 1H), 1.50-1.38 (m, 1H), 1.08 (d, J=6.0 Hz, 3H).

6-chloro-N-((2R,4R)-2-methyltetrahydro-2H-pyran-4-yl)-3-nitroquinolin-4-amine: LCMS [M+H]⁺=322.1. ¹H NMR (400 MHz, CDCl₃) δ ppm: 9.38 (s, 1H), 9.13 (d, J=8.4 Hz, 1H), 8.14 (d, J=2.4 Hz, 1H), 7.99 (d, J=8.8 Hz, 1H), 7.75 (dd, J=2.4, 8.8 Hz, 1H), 4.38-4.22 (m, 1H), 4.17-4.09 (m, 1H), 3.63-3.51 (m, 2H), 2.25-2.11 (m, 2H), 1.86-1.73 (m, 1H), 1.55-1.45 (m, 1H), 1.29 (d, J=6.4 Hz, 3H).

Intermediate 4: LCMS [M+H]⁺=292.1. ¹H NMR (400 MHz, CDCl₃) δ ppm: 8.48 (s, 1H), 7.90 (d, J=8.8 Hz, 1H), 7.74 (d, J=2.0 Hz, 1H), 7.40 (dd, J=2.0, 8.8 Hz, 1H), 4.02 (dd, J=3.6, 11.6 Hz, 1H), 3.89 (br s, 2H), 3.55-3.35 (m, 4H), 1.97-1.78 (m, 2H), 1.62-1.50 (m, 1H), 1.21 (d, J=6.0 Hz, 3H).

### Intermediate 5

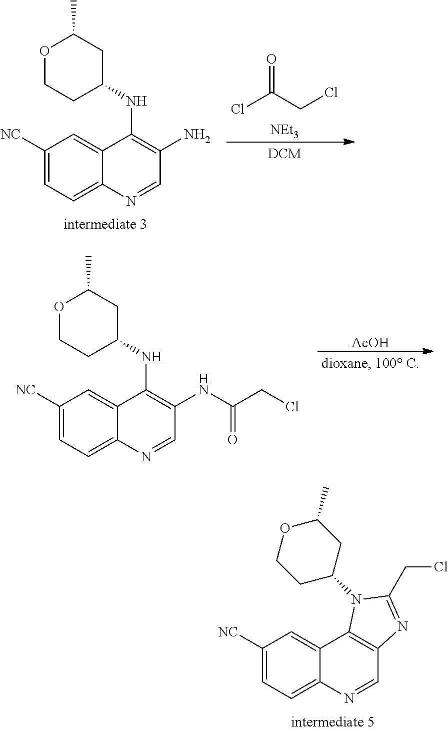

2-chloro-N-(6-cyano-4-(((2R,4R)-2-methyltetrahydro-2H-pyran-4-yl)amino)quinolin-3-yl)acetamide: NEt₃ (739 μL, 5.31 mmol, 3.0 eq) was added to a solution of compound intermediate 3 (500 mg, 1.77 mmol, 1.0 eq) in DCM (10 mL). A solution of chloroacetyl chloride (141 μL, 1.77 mmol, 1.0 eq) in DCM (5 mL) was added dropwise into the reaction mixture at −10° C. The resulting mixture was stirred at −10° C. for 1 h under N₂ atmosphere. The mixture was warmed to 10° C. gradually and stirred for another 1 h under N₂ atmosphere. The reaction mixture was poured into water (20 mL), then extracted with DCM (3×20 mL). The combined organic layers were washed with brine (50 mL), dried over Na₂SO₄, and filtered. The filtrate was concentrated in vacuo. The resulting crude material was purified by silica gel chromatography (90% EtOAc in petroleum ether) to yield the title compound as a yellow solid. LCMS [M+H]⁺=359.1. ¹H NMR (400 MHz, DMSO-d₆) δ ppm: 10.06 (s, 1H), 9.03 (s, 1H), 8.34 (s, 1H), 7.94-7.91 (m, 2H), 6.50 (d, J=8.8 Hz, 1H), 4.35 (s, 2H), 4.12-4.04 (m, 1H), 3.92-3.85 (m, 1H), 3.42-3.39 (m, 2H), 1.90-1.85 (m, 1H), 1.82-1.76 (m, 1H), 1.61-1.51 (m, 1H), 1.20-1.14 (m, 1H), 1.10 (d, J=6.4 Hz, 3H).

Intermediate 5: AcOH (1.0 mL, 17.5 mmol, 20 eq) was added to a solution of 2-chloro-N-(6-cyano-4-(((2R,4R)-2-methyltetrahydro-2H-pyran-4-yl)amino)quinolin-3-yl)acetamide (337 mg, 845 μmol, 1.0 eq) in dioxane (6 mL, 0.1 M) under N₂ atmosphere. The mixture was stirred at 100° C. for 12 h. The reaction was poured into water (20 mL), then extracted with EtOAc (3×20 mL). The combined organic layers were washed with brine (20 mL), dried over Na₂SO₄, and filtered. The filtrate was concentrated in vacuo, and the resulting crude material was purified via silica gel chromatography (100% EtOAc) to yield intermediate 5 as a yellow solid. LCMS [M+H]⁺=341.1. ¹H NMR (400 MHz, DMSO-d₆) δ ppm: 9.39 (s, 1H), 9.01 (br s, 1H), 8.35 (d, J=8.4 Hz, 1H), 8.09 (dd, J=1.6, 8.4 Hz, 1H), 5.33 (br s, 2H), 4.30-4.13 (m, 1H), 3.89-3.66 (m, 2H), 3.40-3.37 (m, 1H), 2.55-2.51 (m, 1H), 2.29-2.01 (m, 3H), 1.26 (d, J=6.4 Hz, 3H).

### Intermediate 6

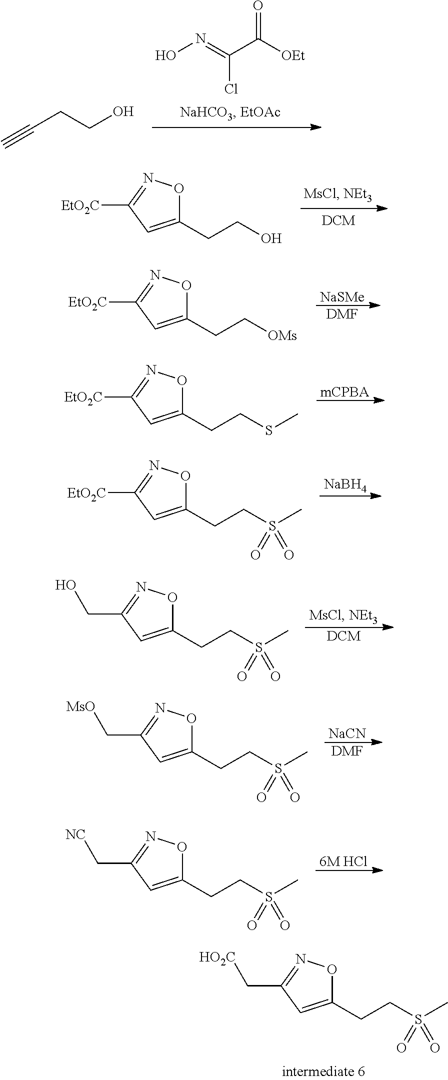

ethyl 5-(2-hydroxyethyl)isoxazole-3-carboxylate: To a solution of homopropargyl alcohol (5.40 mL, 71.34 mmol, 1 eq) and ethyl (chlorohydroximino)acetate (32.43 g, 214 mmol, 3 eq) in EtOAc (500 mL, 0.1 M) was added NaHCO₃ (17.98 g, 214 mmol, 3 eq). The reaction mixture was stirred at 100° C. for 2 h. The mixture was filtered and concentrated in vacuo. The resulting residue was purified via silica gel chromatography (15% EtOAc in petroleum ether) to yield the title compound as a colorless oil. ¹H NMR (400 MHz, CDCl₃) δ ppm 6.52 (s, 1H), 4.39 (q, J=7.2 Hz, 2H), 3.94 (t, J=6.4 Hz, 2H), 3.04 (t, J=6.4 Hz, 2H), 2.64 (br s, 1H), 1.37 (t, J=7.2 Hz, 3H).

ethyl 5-(2-((methylsulfonyl)oxy)ethyl)isoxazole-3-carboxylate: A mixture of ethyl 5-(2-hydroxyethyl)isoxazole-3-carboxylate (4 g, 20.5 mmol, 1 eq) and MsCl (2.05 mL, 26.5 mmol, 1.29 eq) in DCM (80 mL, 0.25 M) was cooled to 0° C. Et₃N (8.57 mL, 61.5 mmol, 3 eq) was added dropwise, and the mixture was warmed to room temperature over 2.5 h. The reaction mixture was then slowly poured into ice-water (100 mL), and the mixture was extracted with DCM (2×100 mL). The combined organic layers were washed with brine (2×100 mL). The organic layer was dried over Na₂SO₄, filtered and concentrated in vacuo to yield the title compound as a yellow oil, which was used for the next step directly without further purification. LCMS [M+H]⁺=264.0. ¹H NMR (400 MHz, CDCl₃) δ ppm 6.61-6.57 (m, 1H), 4.56-4.49 (m, 2H), 4.47-4.38 (m, 2H), 3.33-3.25 (m, 2H), 3.02-2.98 (m, 3H), 1.44-1.38 (m, 3H).

ethyl 5-(2-(methylthio)ethyl)isoxazole-3-carboxylate: A mixture of sodium methane thiolate (2.64 mL, 41.5 mmol, 2 eq) in DMF (60 mL) was sealed with rubber septum in a round bottom flask and placed under N₂ atmosphere. Ethyl 5-(2-((methylsulfonyl)oxy)ethyl)isoxazole-3-carboxylate (5.46 g, 20.7 mmol, 1 eq) in DMF (100 mL) was added to the reaction mixture, then the mixture was stirred at 80° C. for 6 hours. The reaction mixture was poured into water (200 mL) and extracted with EtOAc (3×200 mL). The combined organic layers were washed with brine (3×200 mL). The organic layer was dried over Na₂SO₄, filtered and concentrated in vacuo to yield the title compound as a yellow oil, which was used for the next step directly without further purification. LCMS [M+H]⁺=216.1. ¹H NMR (400 MHz, CDCl₃) δ ppm 6.50 (s, 1H), 4.42 (q, J=7.2 Hz, 2H), 3.10 (t, J=7.2 Hz, 2H), 2.83 (t, J=7.2 Hz, 2H), 2.12 (s, 3H), 1.40 (t, J=7.2 Hz, 3H).

ethyl 5-(2-(methylsulfonyl)ethyl)isoxazole-3-carboxylate: To a solution of ethyl 5-(2-(methylthio)ethyl)isoxazole-3-carboxylate (1.6 g, 6.47 mmol, 1 eq) in DCE (50 mL, 0.1 M) was added m-CPBA (4.18 g, 19.4 mmol, 3 eq) portion-wise at 0° C. The reaction mixture was stirred at 85° C. for 3 h. The reaction mixture was poured into a 1:1 mixture of saturated of NaHCO₃ (100 mL) and saturated Na₂SO₃ (100 mL). The solution was extracted with DCM (3×100 mL), and the combined organic layers were washed with brine (3×200 mL). The organic layer was dried over Na₂SO₄, filtered and concentrated in vacuo to yield the title compound as a yellow solid, which was used for the next step without further purification. LCMS [M+H]+=248.0. ¹H NMR (400 MHz, CDCl₃) δ ppm 6.59 (s, 1H), 4.44 (q, J=7.2 Hz, 2H), 3.45-3.40 (m, 4H), 2.95 (s, 3H), 1.41 (t, J=7.2 Hz, 3H).

(5-(2-(methylsulfonyl)ethyl)isoxazol-3-yl)methanol: To a solution of ethyl 5-(2-(methylsulfonyl)ethyl)isoxazole-3-carboxylate (900 mg, 3.64 mmol, 1 eq) in MeOH (10 mL, 0.4 M) was added NaBH₄ (1.38 g, 36.4 mmol, 10 eq) slowly at 0° C. The reaction mixture was stirred at 20° C. for 12 h under N₂ atmosphere. The mixture was quenched with H₂O (5 mL) and sat. NH₄Cl (5 mL), and the reaction mixture was concentrated in vacuo. The resulting crude material was purified via silica gel chromatography (3% MeOH in DCM) to yield the title compound as a white solid. ¹H NMR (400 MHz, DMSO-d₆) δ ppm 6.37 (s, 1H), 5.47 (t, J=6.0 Hz, 1H), 4.46 (d, J=6.0 Hz, 2H), 3.57-3.50 (m, 2H), 3.23-3.16 (m, 2H), 3.03 (s, 3H).

(5-(2-(methylsulfonyl)ethyl)isoxazol-3-yl)methyl methanesulfonate: To a solution of (5-(2-(methylsulfonyl)ethyl)isoxazol-3-yl)methanol (930 mg, 4.08 mmol, 1 eq) in DCM (20 mL, 0.2 M) was added TEA (1.70 mL, 12.2 mmol, 3 eq) and MsCl (574 μL, 7.42 mmol, 1.82 eq) slowly at 0° C. The reaction mixture was stirred at 20° C. for 1 h under N₂ atmosphere. The reaction mixture was slowly poured into ice-water (50 mL) and extracted with DCM (2×50 mL). The combined organic layers were washed with brine (2×50 mL). The organic layer was dried over Na₂SO₄, filtered and concentrated in vacuo to give the title compound as a white solid, which was used for the next step directly without further purification. LCMS [M+H]⁺=284.0. ¹H NMR (400 MHz, DMSO-d₆) δ ppm 6.57 (s, 1H), 5.33 (s, 2H), 3.59-3.54 (m, 2H), 3.28 (s, 3H), 3.28-3.23 (m, 2H), 3.03 (s, 3H).

2-(5-(2-(methylsulfonyl)ethyl)isoxazol-3-yl)acetonitrile: To a solution of (5-(2-(methylsulfonyl)ethyl)isoxazol-3-yl)methyl methanesulfonate (1.07 g, 3.78 mmol, 1 eq) in DMF (15 mL, 0.25 M) was added NaCN (277 mg, 5.65 mmol, 1.5 eq) at 0° C. The reaction mixture was stirred at 20° C. for 2 h under N₂ atmosphere. The reaction mixture was slowly poured into ice-water (50 mL), and the mixture was extracted with DCM (2×50 mL). The combined organic layers were washed with brine (2×50 mL). The organic layer was dried over Na₂SO₄, filtered and concentrated in vacuo. The resulting crude material was purified via silica gel chromatography (5% MeOH in DCM) to yield the title compound as a yellow oil. CAUTION: 2 M NaOH aq. was added to the separated aqueous phase until pH=14 and the mixture was quenched with sat. aq. NaClO (100 mL). LCMS [M+H]⁺=215.0. ¹H NMR (400 MHz, DMSO-d₆) δ ppm 6.48 (s, 1H), 4.19 (s, 2H), 3.58-3.52 (m, 2H), 3.27-3.21 (m, 2H), 3.03 (s, 3H).

Intermediate 6: To a solution of 2-(5-(2-(methylsulfonyl)ethyl)isoxazol-3-yl)acetonitrile (150 mg, 595 μmol, 1 eq) in H₂O (0.5 mL) was added a solution of conc. HCl (2 mL, 20.1 mmol, 33 eq). The reaction mixture was stirred at 90° C. for 7 h under N₂ atmosphere. The reaction mixture was lyophilized directly to give example 6 as a white solid. The crude product was used for the next step directly without further purification. ¹H NMR (400 MHz, DMSO-d₆) δ ppm 12.67 (s, 1H), 6.38 (s, 1H), 3.66 (s, 2H), 3.53 (t, J=6.4 Hz, 2H), 3.20 (t, J=6.4 Hz, 2H), 3.02 (s, 3H).

### Intermediate 7

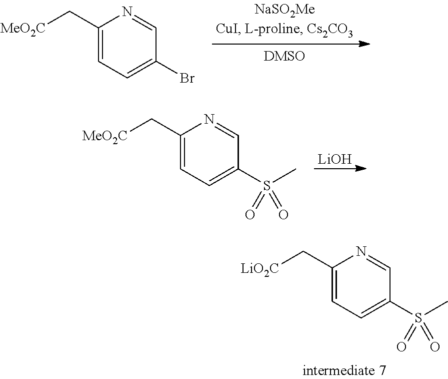

methyl 2-(5-(methylsulfonyl)pyridin-2-yl)acetate: To a solution of methyl 2-(5-bromopyridin-2-yl)acetate (500 mg, 2.17 mmol, 1 eq) in DMSO (4 mL, 0.5 M) was added L-proline (300.26 mg, 2.61 mmol, 1.2 eq), Cs₂CO₃ (495.69 mg, 1.52 mmol, 0.7 eq), CuI (290 mg, 1.52 mmol, 0.7 eq) and sodium methanesulfinate (333 mg, 3.26 mmol, 1.5 eq). The mixture was bubbled with N₂ and stirred at 100° C. for 6 h under microwave (2 bar). The mixture was quenched with water (50 mL) and extracted with EtOAc (3×50 mL). The combined organic layers were washed with brine (2×50 mL). The organic layer was dried over Na₂SO₄, filtered and concentrated in vacuo. The resulting crude material was purified via silica gel chromatography (0-100% EtOAc in petroleum ether) to yield the title compound as a yellow solid. LCMS [M+H]⁺=230.0. ¹H NMR (400 MHz, CDCl₃) δ ppm: 9.09 (s, 1H), 8.21 (dd, J=2.0, 8.0 Hz, 1H), 7.54 (d, J=8.0 Hz, 1H), 3.99 (s, 2H), 3.76 (s, 3H), 3.12 (s, 3H).

Intermediate 7: To a solution of methyl 2-(5-(methylsulfonyl)pyridin-2-yl)acetate (180 mg, 785 μmol, 1 eq) in THF (3 mL) and H₂O (3 mL) was added LiOH·H₂O (65.90 mg, 1.57 mmol, 2 eq) at 0° C. Then the mixture was stirred at 20° C. for 2 h. The reaction mixture was slowly poured into water (5 mL) and extracted with EtOAc (1×5 mL). The aqueous phase was purified directly via RP-chromatography (0% MeCN in water) to yield intermediate 7 as a white solid. ¹H NMR (400 MHz, DMSO-d₆) δ ppm 8.88-8.82 (m, 1H), 8.16-8.09 (m, 1H), 7.59-7.51 (m, 1H), 3.55-3.49 (m, 2H), 3.27 (s, 3H).

### Intermediate 8

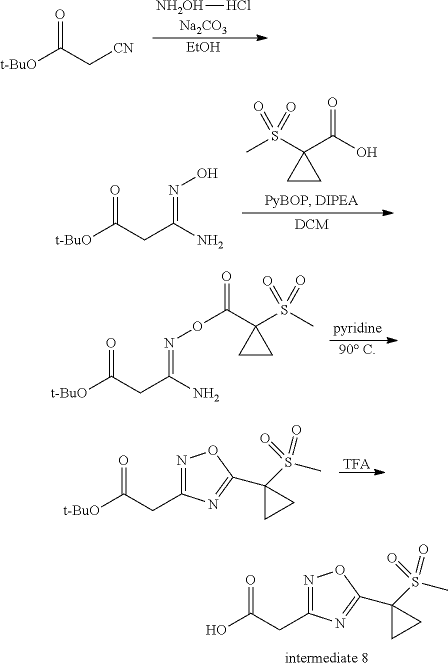

tert-butyl (Z)-3-amino-3-(hydroxyimino)propanoate: Water (6 mL) was added dropwise to a vigorously stirred mixture of cyanoacetic acid tert-butyl ester (1.0 mL, 7.08 mmol, 1 eq), NH₂OH·HCl (0.5 mL, 10.6 mmol, 1.5 eq) and Na₂CO₃ (578 mg, 5.45 mmol, 0.77 eq) in EtOH (10 mL) at 25° C. The resulting solution was stirred at room temperature for 10 h. The mixture was concentrated in vacuo, diluted with water (10 mL), and extracted with EtOAc (2×10 mL). The combined organic layers were washed with brine (2×10 mL). The organic layer was dried over Na₂SO₄, filtered and concentrated in vacuo to give the title compound as an off-white solid, which was used in the next step without further purification. LCMS [M+H]⁺=175.1. ¹H NMR (400 MHz, DMSO-d₆) δ ppm: 8.98 (s, 1H), 5.41 (s, 2H), 2.90 (s, 2H), 1.40 (s, 9H).

tert-butyl (Z)-3-amino-3-(((1-(methylsulfonyl)cyclopropane-1-carbonyl)oxy)imino)propanoate: To a solution of 1-(methylsulfonyl)cyclopropanecarboxylic acid (500 mg, 3.05 mmol, 1 eq) and tert-butyl (Z)-3-amino-3-(hydroxyimino)propanoate (589 mg, 3.05 mmol, 1 eq) in DCM (15 mL, 0.2 M) was added PyBOP (1.58 g, 3.05 mmol, 1 eq) and DIPEA (2.12 mL, 12.2 mmol, 4 eq) at 25° C. Then the mixture was stirred at 25° C. for 12 h. The mixture quenched with water (40 mL) and extracted with DCM (2×40 mL). The combined organic layers were washed with brine (2×40 mL). The organic layer was dried over Na₂SO₄, filtered and concentrated in vacuo. The resulting crude material was purified via silica gel chromatography (10-50% EtOAc in petroleum ether) to provide the title compound as a brown oil. LCMS [M+H]⁺=321.0. ¹H NMR (400 MHz, DMSO-d₆) δ ppm: 6.91-6.17 (m, 2H), 3.25 (s, 3H), 3.08 (s, 2H), 1.80-1.73 (m, 2H), 1.66-1.60 (m, 2H), 1.42 (s, 9H).

tert-butyl 2-(5-(1-(methylsulfonyl)cyclopropyl)-1,2,4-oxadiazol-3-yl)acetate: A solution of tert-butyl (Z)-3-amino-3-(((1-(methylsulfonyl)cyclopropane-1-carbonyl)oxy)imino)propanoate (650 mg, 1.83 mmol, 1 eq) in pyridine (7 mL, 87 mmol, 48 eq) was stirred at 90° C. for 12 h under N₂ atmosphere. The mixture was concentrated in vacuo, and the resulting crude material was purified by prep-HPLC (NH₄HCO₃ conditions) to yield the title compound as a brown oil. LCMS [M−56+H]+=247.0. ¹H NMR (400 MHz, CDCl₃) δ ppm: 3.76 (s, 2H), 3.32 (s, 3H), 2.11-2.05 (m, 2H), 1.86-1.81 (m, 2H), 1.48 (s, 9H).

Intermediate 8: To a solution of tert-butyl 2-(5-(1-(methylsulfonyl)cyclopropyl)-1,2,4-oxadiazol-3-yl)acetate (90 mg, 267 μmol, 1 eq) in DCM (2 mL) was added TFA (2 mL, 27 mmol, 100 eq). The mixture was stirred at 25° C. for 6 h. The solution was concentrated in vacuo to yield intermediate 8 as a brown oil, which was used directly in the next step without further purification. ¹H NMR (400 MHz, CDCl₃) δ ppm: 3.92 (s, 2H), 3.32 (s, 3H), 2.13-2.05 (m, 2H), 1.89-1.82 (m, 2H).

### Intermediate 9

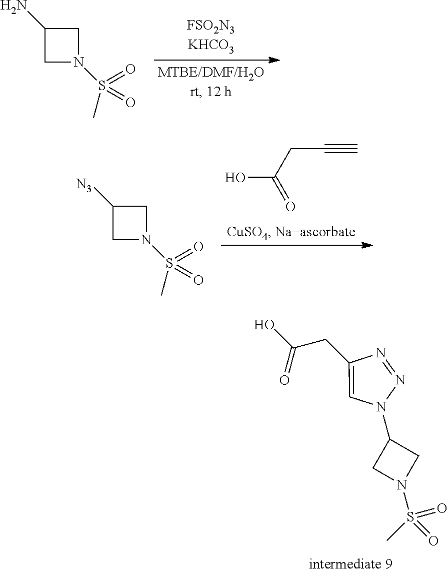

3-azido-1-(methylsulfonyl)azetidine: To a 50 mL roundbottom flask containing DMF (0.76 mL) and aqueous KHCO₃ (3 M, 887 μL, 4 eq) was added 1-methylsulfonylazetidin-3-amine (100 mg, 665 μmol, 1 eq) and fluorosulfuryl azide (0.26 M in MTBE, 2.56 mL, 1 eq). The reaction mixture was stirred at room temperature for 12 h. The reaction mixture was then diluted with EtOAc (20 mL) and washed with brine (3×10 mL). The organic layer was dried over Na₂SO₄, filtered and concentrated in vacuo to afford the title compound, which was used in the next step without further purification. ¹H NMR (400 MHz, DMSO-d₆) δ ppm: 4.48 (tt, J=5.2, 7.2 Hz, 1H), 4.17-4.08 (m, 2H), 3.79 (dd, J=5.2, 9.6 Hz, 2H).

Intermediate 9: CuSO₄ (1 M in water, 178.78 μL, 0.3 eq) and sodium L-ascorbate (1 M in water, 178 μL, 0.3 eq) were added to a solution of 3-azido-1-(methylsulfonyl)azetidine (105 mg, 595 μmol, 1 eq), citric acid (125 mg, 595 μmol, 1 eq) and 3-butynoic acid (50.1 mg, 595 μmol, 1 eq) in t-BuOH (2 mL), DMSO (2 mL), and H₂O (2 mL) under N₂ atmosphere. The reaction mixture was stirred at 20° C. for 12 h. The solution was then extracted with DCM (3×10 mL) and EtOAc (1×10 mL). The aqueous layer was lyophilized under reduced pressure to give a pink solid, which was dissolved with DCM (100 mL). The solution was filtered, and the filtrate was concentrated in vacuo to give the title compound, which was used in the next step without further purification. ¹H NMR (500 MHz, CDCl₃) δ ppm: 8.18 (br s, 1H), 5.62-5.47 (m, 1H), 4.45-4.35 (m, 2H), 4.33-4.24 (m, 2H), 3.71 (s, 2H), 3.14 (s, 3H).

### Example 1. Procedure A: Synthesis of Compound 1

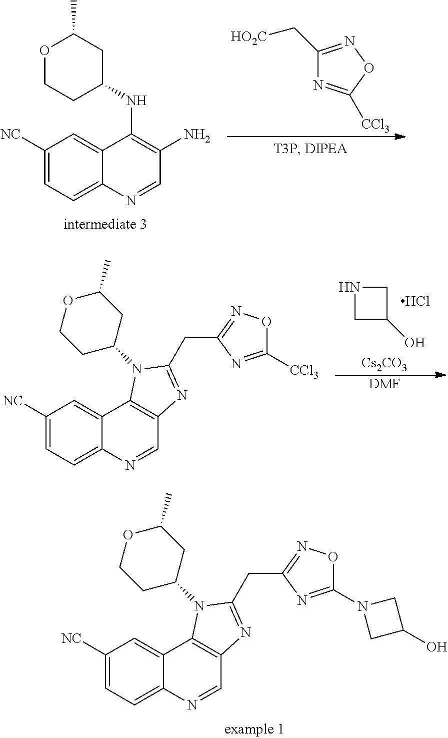

1-((2R,4R)-2-methyltetrahydro-2H-pyran-4-yl)-2-((5-(trichloromethyl)-1,2,4-oxadiazol-3-yl)methyl)-1H-imidazo[4,5-c]quinoline-8-carbonitrile: DIPEA (416 μL, 2.39 mmol, 3 eq) was added to a microwave vessel charged with intermediate 4 (225 mg, 796 μmol, 1 eq), 2-(5-(trichloromethyl)-1,2,4-oxadiazol-3-yl) acetic acid (254 mg, 1.04 mmol, 1.3 eq), T3P (50% in EtOAc, 1.14 mL, 1.91 mmol, 2.4 eq) and EtOAc (2 mL, 0.4 M) under N₂ atmosphere at 0° C. The reaction mixture was irradiated with a microwave reactor for 13 h (85° C., 2 bar). After cooling to 20° C., the mixture was diluted with DCM (70 mL) and washed with brine (3×15 mL). The organic layer was dried over Na₂SO₄, filtered and concentrated in vacuo. The resulting residue was purified via silica gel chromatography (50% EtOAc in petroleum ether) to yield the title compound as an orange solid. LCMS [M+H]⁺=491.1. ¹H NMR (400 MHz, DMSO-d₆) δ ppm 9.35 (s, 1H), 9.03 (br s, 1H), 8.34 (d, J=8.4 Hz, 1H), 8.08 (d, J=7.6 Hz, 1H), 5.48-5.22 (m, 1H), 5.01 (s, 2H), 4.19 (br s, 1H), 3.71 (br s, 2H), 2.21 (br s, 2H), 2.13 (br s, 2H), 1.24 (d, J=6.0 Hz, 3H).

2-{[5-(3-hydroxyazetidin-1-yl)-1,2,4-oxadiazol-3-yl]methyl}-1-[(2R,4R)-2-methyloxan-4-yl]-1H-imidazo[4,5-c]quinoline-8-carbonitrile (Compound 1): Cs₂CO₃ (53.7 mg, 164 μmol, 3 eq) was added to a solution of 1-((2R,4R)-2-methyltetrahydro-2H-pyran-4-yl)-2-((5-(trichloromethyl)-1,2,4-oxadiazol-3-yl)methyl)-1H-imidazo[4,5-c]quinoline-8-carbonitrile (30 mg, 54.9 μmol, 1 eq) and azetion-3-ol hydrochloride (18.1 mg, 164 μmol, 3 eq) in DMF (1 mL, 0.05 M) under N₂ atmosphere. The reaction mixture was stirred at 20° C. for 12 h. The mixture was diluted with water (20 mL) and extracted with EtOAc (3×10 mL). The combined organic layers were dried over Na₂SO₄, filtered and concentrated in vacuo. The resulting residue was purified by prep-HPLC (NH₄OH conditions) to give Compound 1 as a white solid. LCMS [M+H]⁺=446.2. ¹H NMR (400 MHz, DMSO-d₆) δ ppm 9.35 (s, 1H), 9.02 (br s, 1H), 8.34 (d, J=8.8 Hz, 1H), 8.06 (d, J=8.4 Hz, 1H), 5.90 (d, J=6.8 Hz, 1H), 5.32 (br s, 1H), 4.63-4.58 (m, 1H), 4.56 (br s, 2H), 4.36-4.30 (m, 2H), 4.26-4.14 (m, 1H), 3.90 (dd, J=4.4, 8.8 Hz, 2H), 3.83-3.58 (m, 2H), 2.42-2.38 (m, 1H), 2.17 (br s, 2H), 2.09-1.99 (m, 1H), 1.24 (d, J=6.0 Hz, 3H).

### Example 2. Procedure B: Synthesis of Compound 2

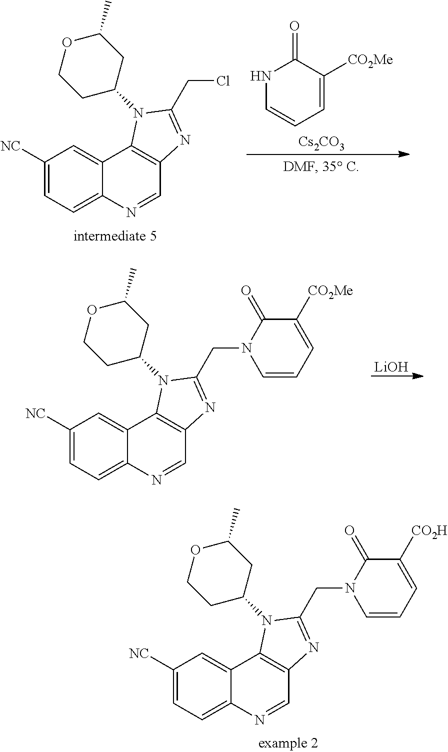

methyl 1-((8-cyano-1-((2R,4R)-2-methyltetrahydro-2H-pyran-4-yl)-1H-imidazo[4,5-c]quinolin-2-yl)methyl)-2-oxo-1,2-dihydropyridine-3-carboxylate: A mixture of intermediate 5 (10 mg, 29.3 μmol, 1 eq), methyl 2-hydroxynicotinate (8.99 mg, 58.7 μmol, 2 eq) and Cs₂CO₃ (28.7 mg, 88.0 μmol, 3 eq) in DMF (2 mL, 0.01 M) was stirred at 35° C. for 12 h. The mixture was filtered, and the filtrate was purified by prep-HPLC (NH₄HCO₃ conditions) to yield the title compound as a white solid. LCMS [M+H]⁺=458.2. ¹H NMR (400 MHz, DMSO-d₆) δ ppm: 9.28 (s, 1H), 9.01 (s, 1H), 8.31 (d, J=8.4 Hz, 1H), 8.22 (dd, J=2.0, 6.8 Hz, 1H), 8.15 (dd, J=2.0, 7.2 Hz, 1H), 8.05 (dd, J=1.6, 8.4 Hz, 1H), 6.49 (t, J=6.8 Hz, 1H), 5.69 (s, 2H), 5.60-5.41 (m, 1H), 4.23-4.10 (m, 1H), 3.94-3.75 (m, 2H), 3.71 (s, 3H), 2.47-2.36 (m, 1H), 2.30-2.05 (m, 3H), 1.27 (d, J=6.4 Hz, 3H).

1-({8-cyano-1-[(2R,4R)-2-methyloxan-4-yl]-1H-imidazo[4,5-c]quinolin-2-yl}methyl)-2-oxo-1,2-dihydropyridine-3-carboxylic acid (Compound 2): A mixture of methyl 1-((8-cyano-1-((2R,4R)-2-methyltetrahydro-2H-pyran-4-yl)-1H-imidazo[4,5-c]quinolin-2-yl)methyl)-2-oxo-1,2-dihydropyridine-3-carboxylate (67 mg, 146 μmol, 1 eq) and LiOH·H₂O (30.7 mg, 732 μmol, 5 eq) in THF (5 mL, 0.03 M) was stirred at 25° C. for 12 h. The mixture was adjusted to pH=3 with 1N HCl and concentrated in vacuo. The resulting residue was purified by prep-HPLC (NH₄HCO₃ conditions) to yield example 2 as a white solid. LCMS [M+H]⁺=444.2. ¹H NMR (400 MHz, DMSO-d₆) δ ppm: 14.03 (s, 1H), 9.28 (s, 1H), 9.02 (s, 1H), 8.53-8.47 (m, 1H), 8.45-8.40 (m, 1H), 8.31 (d, J=8.8 Hz, 1H), 8.07 (dd, J=1.6, 8.8 Hz, 1H), 6.87 (t, J=6.8 Hz, 1H), 5.91 (s, 2H), 5.53 (br s, 1H), 4.17 (br s, 1H), 3.97-3.76 (m, 2H), 2.46-2.39 (m, 1H), 2.31-2.05 (m, 3H), 1.27 (d, J=6.0 Hz, 3H).

### Example 3. Procedure C: Synthesis of Compound 3

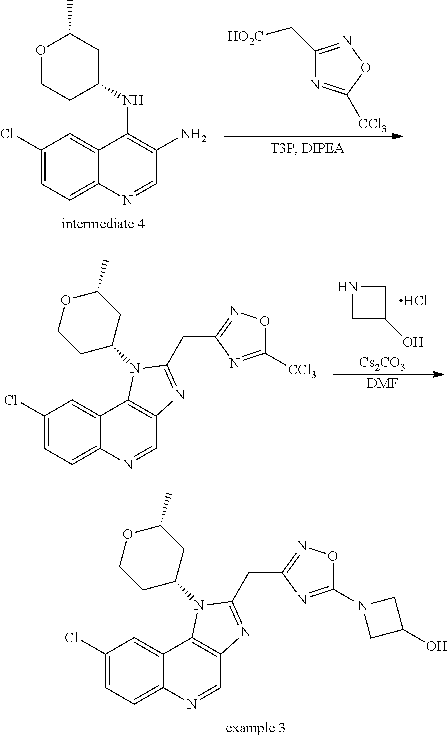

The compound of Example 3 was prepared via the same method as example 1 using intermediate 4 as the starting material.

3-((8-chloro-1-((2R,4R)-2-methyltetrahydro-2H-pyran-4-yl)-1H-imidazo[4,5-c]quinolin-2-yl)methyl)-5-(trichloromethyl)-1,2,4-oxadiazole: LCMS [M+H]+=502.0.

1-[3-({8-chloro-1-[(2R,4R)-2-methyloxan-4-yl]-1H-imidazo[4,5-c]quinolin-2-yl}methyl)-1,2,4-oxadiazol-5-yl]azetidin-3-ol (Compound 3): LCMS [M+H]⁺=455.2. ¹H NMR (400 MHz, DMSO-d₆) δ ppm 9.20 (s, 1H), 8.68 (br s, 1H), 8.20 (d, J=8.8 Hz, 1H), 7.76 (dd, J=2.0, 8.8 Hz, 1H), 5.90 (d, J=6.4 Hz, 1H), 5.24 (br s, 1H), 4.64-4.57 (m, 1H), 4.53 (s, 2H), 4.32 (t, J=7.8 Hz, 2H), 4.22-4.13 (m, 1H), 3.90 (dd, J=4.4, 8.8 Hz, 2H), 3.76-3.58 (m, 2H), 2.47-2.38 (m, 1H), 2.27-2.11 (m, 2H), 2.06-1.97 (m, 1H), 1.23 (d, J=6.0 Hz, 3H).

### Example 4. Procedure D: Synthesis of Compound 4

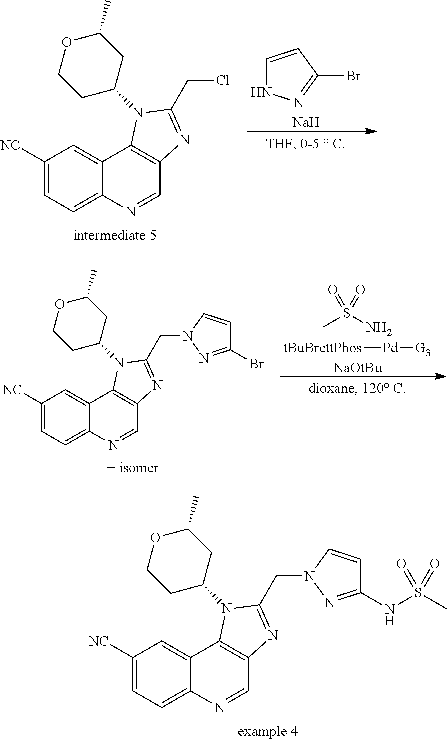

2-((3-bromo-1H-pyrazol-1-yl)methyl)-1-((2R,4R)-2-methyltetrahydro-2H-pyran-4-yl)-1H-imidazo[4,5-c]quinoline-8-carbonitrile: To a solution of 3-bromo-1H-pyrazole (63.1 mg, 429 μmol, 1.5 eq) in THF (5 mL) was added NaH (60% in mineral oil, 22.9 mg, 572 μmol, 2 eq) at 0° C. The reaction mixture was stirred at 0° C. for 30 min. After this time, a solution of intermediate 5 (100 mg, 286 μmol, 1 eq) in THF (3 mL) was added dropwise. The reaction mixture was then stirred at 0-5° C. for another 2 h. After this time, the mixture was poured into ice-water (10 mL) slowly and the mixture was extracted with DCM (2×10 mL). The combined organic layers were washed with brine (2×20 mL), dried over Na₂SO₄, filtered and concentrated in vacuo. The resulting crude mixture of isomers was purified via silica gel chromatography (80-100% EtOAc in petroleum ether) to give the title compound as a yellow solid. LCMS [M+H]⁺=451.0/453.0. ¹H NMR (500 MHz, DMSO-d₆) δ ppm 9.42-9.34 (m, 1H), 8.97 (br s, 1H), 8.34 (d, J=9.0 Hz, 1H), 8.07 (d, J=8.5 Hz, 1H), 8.00 (br s, 1H), 6.50 (d, J=2.5 Hz, 1H), 5.99 (s, 2H), 5.50-5.28 (m, 1H), 4.24-4.10 (m, 1H), 3.81-3.59 (m, 2H), 2.48-2.39 (m, 1H), 2.22-2.05 (m, 1H), 2.02-1.69 (m, 2H), 1.23 (d, J=6.0 Hz, 3H).

N-(1-{[8-cyano-1-(2-methyloxan-4-yl)-1H-imidazo[4,5-c]quinolin-2-yl]methyl}-1H-pyrazol-3-yl)methanesulfonamide (Compound 4): To a solution of 2-((3-bromo-1H-pyrazol-1-yl)methyl)-1-((2R,4R)-2-methyltetrahydro-2H-pyran-4-yl)-1H-imidazo[4,5-c]quinoline-8-carbonitrile (40 mg, 82.7 μmol, 1 eq) and methanesulfonamide (78.7 mg, 826 μmol, 10 eq) in dioxane (2 mL, 0.04 M) was added t-BuBrettPhos Pd G3 (7.1 mg, 8.27 μmol, 0.1 eq), tBuONa (23.8 mg, 248 μmol, 3 eq) in sequence under N₂ atmosphere. The reaction mixture was heated to 120° C. and stirred for 16 hours under N₂ atmosphere. The reaction mixture was concentrated in vacuo. The resulting residue was diluted with DCM (10 mL) and ice-water (10 mL). The mixture was extracted with DCM (2×20 mL). The combined organic layers were washed with brine (2×20 mL), dried over Na₂SO₄, filtered and concentrated in vacuo. The resulting crude material was purified by prep-HPLC (NH₄HCO₃ conditions) to yield example 4 as a white solid. LCMS [M+H]⁺=466.1. ¹H NMR (400 MHz, DMSO-d₆) δ ppm 9.79 (br s, 1H), 9.38 (s, 1H), 8.98 (br s, 1H), 8.34 (d, J=8.8 Hz, 1H), 8.07 (dd, J=1.2, 8.8 Hz, 1H), 7.89 (br s, 1H), 6.09 (d, J=2.4 Hz, 1H), 5.88 (s, 2H), 5.46-5.24 (m, 1H), 4.25-4.04 (m, 1H), 3.79-3.56 (m, 2H), 2.96 (s, 3H), 2.46-2.35 (m, 1H), 2.19-2.03 (m, 1H), 2.02-1.68 (m, 2H), 1.22 (d, J=6.0 Hz, 3H).

### Example 5. Procedure E: Synthesis of Compound 5

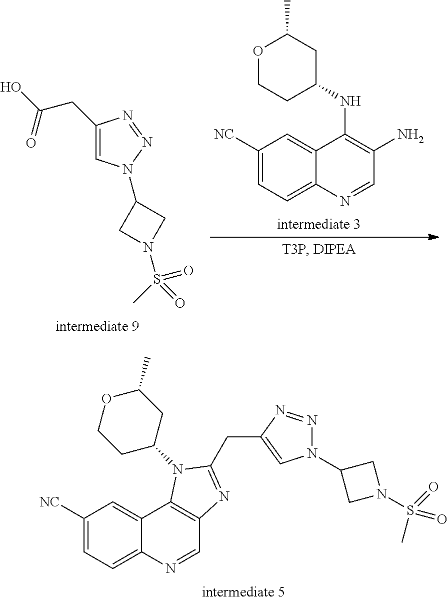 [SMILES: CS(=O)(=O)N1CC(n2cc(CC(=O)O)nn2)C1 ; C[C@@H]1C[C@H](Nc2c(N)cnc3ccc(C#N)cc23)CCO1 ; C[C@@H]1C[C@H](n2c(Cc3cn(C4CN(S(C)(=O)=O)C4)nn3)nc3cnc4ccc(C#N)cc4c32)CCO1]

2-{[1-(1-methanesulfonylazetidin-3-yl)-1H-1,2,3-triazol-4-yl]methyl}-1-[(2R,4R)-2-methyloxan-4-yl]-1H-imidazo[4,5-c]quinoline-8-carbonitrile (Compound 5): T3P (50% in EtOAc, 205 μL, 346 μmol, 3 eq) was added to a solution of intermediate 9 (30 mg, 115 μmol, 1 eq), intermediate 3 (32.5 mg, 115 μmol, 1 eq) and DIPEA (200 μL, 1.15 mmol, 10 eq) in DMF (2 mL) under N₂ atmosphere. The mixture was stirred at 20° C. for 1 h, then 130° C. for 3 h. The mixture was poured into water (10 mL) and extracted with EtOAc (3×20 mL). The combined organic layers were dried over Na₂SO₄, filtered and concentrated in vacuo. The resulting residue was purified by prep HPLC (NH₄HCO₃ conditions) to give example 5 as a white solid. LCMS [M+H]⁺=507.2. ¹H NMR (500 MHz, DMSO-d₆) δ ppm: 9.37 (s, 1H), 9.02 (br s, 1H), 8.34 (d, J=8.5 Hz, 1H), 8.32 (s, 1H), 8.07 (dd, J=1.5, 8.5 Hz, 1H), 5.60-5.51 (m, 1H), 5.48-5.32 (m, 1H), 4.71 (s, 2H), 4.40 (t, J=8.5 Hz, 2H), 4.29 (dd, J=6.0, 9.0 Hz, 2H), 4.17 (s, 1H), 3.81-3.61 (m, 2H), 3.13 (s, 3H), 2.49-2.39 (m, 1H), 2.25-1.84 (m, 3H), 1.23 (d, J=6.0 Hz, 3H).

### Example 6. Procedure F: Synthesis of Compound 6

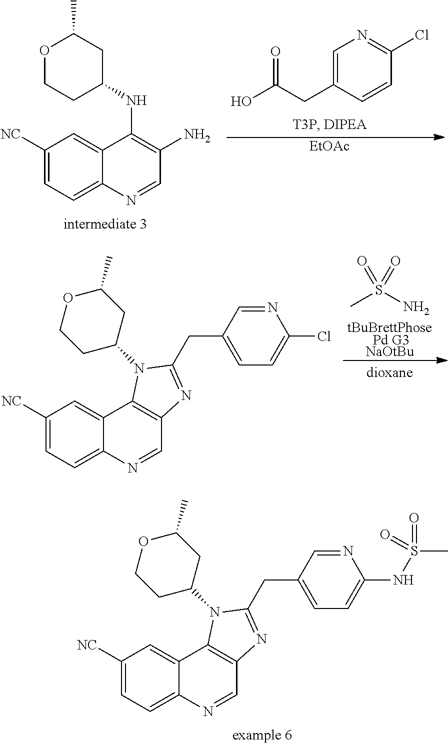

2-((6-chloropyridin-3-yl)methyl)-1-((2R,4R)-2-methyltetrahydro-2H-pyran-4-yl)-1H-imidazo[4,5-c]quinoline-8-carbonitrile: T3P (50% in EtOAc, 1.01 mL, 1.70 mmol, 2.4 eq) was added to a microwave vessel charged with intermediate 3 (200 mg, 708 μmol, 1 eq), 2-(6-chloropyridin-3-yl)acetic acid (145.85 mg, 850 μmol, 1.2 eq), DIPEA (370 μL, 2.13 mmol, 3 eq) and EtOAc (7 mL, 0.1 M) under N₂ atmosphere at 0° C. The reaction mixture was irradiated with a microwave reactor for 10 h (85° C., 2 bar). The mixture was diluted with DCM (50 mL) and washed with brine (3×15 mL). The organic phase was dried over Na₂SO₄, filtered and concentrated in vacuo. The resulting residue was purified by prep-HPLC (HCl conditions) to give the title compound as a yellow solid. LCMS [M+H]⁺=418.1.

N-(5-{[8-cyano-1-(2-methyloxan-4-yl)-1H-imidazo[4,5-c]quinolin-2-yl]methyl}pyridin-2-yl)methanesulfonamide (Compound 6): To a solution of 2-((6-chloropyridin-3-yl)methyl)-1-((2R,4R)-2-methyltetrahydro-2H-pyran-4-yl)-1H-imidazo[4,5-c]quinoline-8-carbonitrile (50 mg, 87.34 μmol, 1 eq) and methanesulfonamide (83.1 mg, 873 μmol, 10 eq) in dioxane (2 mL, 0.04 M) was added tBuBrettPhos Pd G3 (7.46 mg, 8.73 μmol, 0.1 eq), t-BuONa (25.2 mg, 262 μmol, 3 eq) in sequence under N₂ atmosphere. The reaction mixture was then heated to 120° C. and stirred for 16 h under N₂ atmosphere. The reaction mixture was diluted with EtOAc (50 mL) and washed with brine (3×10 mL). The organic layer was dried over Na₂SO₄, filtered and concentrated in vacuo. The resulting residue was purified with prep-HPLC (NH₄OH conditions) to give example 6 as a white solid. LCMS [M+H]⁺=477.2. ¹H NMR (400 MHz, DMSO-d₆) δ ppm 10.56 (br s, 1H), 9.31 (s, 1H), 8.97 (s, 1H), 8.35-8.21 (m, 2H), 8.03 (dd, J=1.2, 8.4 Hz, 1H), 7.69 (dd, J=2.0, 8.4 Hz, 1H), 6.97 (d, J=7.6 Hz, 1H), 5.43-5.15 (m, 1H), 4.53 (s, 2H), 4.19-4.10 (m, 1H), 3.25 (br s, 3H), 2.22-2.02 (m, 2H), 1.89 (br s, 2H), 1.21 (d, J=6.1 Hz, 3H).

### Example 7. Procedure G: Synthesis of Compound 7

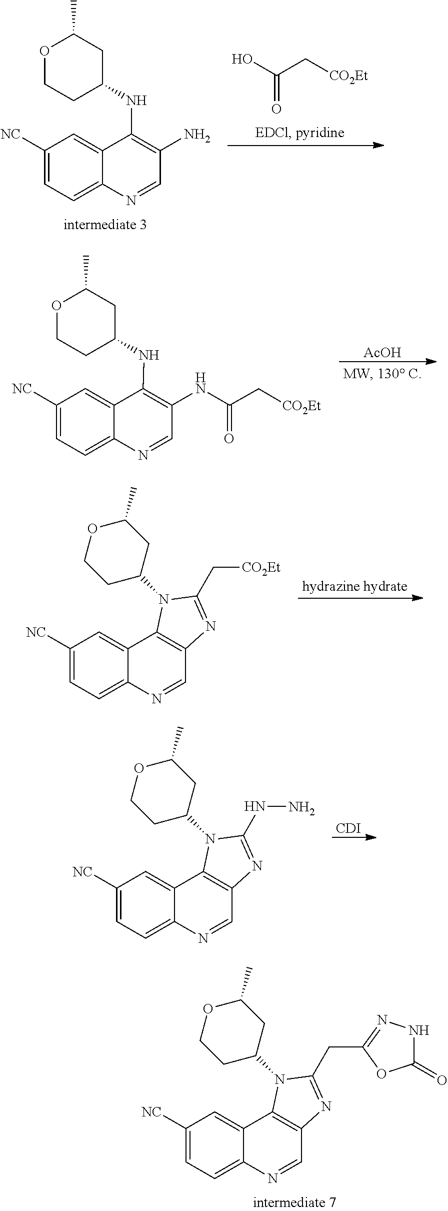

ethyl 3-((6-cyano-4-(((2R,4R)-2-methyltetrahydro-2H-pyran-4-yl)amino)quinolin-3-yl)amino)-3-oxopropanoate: To a solution of compound intermediate 3 (400 mg, 1.42 mmol, 1 eq), monoethyl malonate (251 mg, 2.13 mmol, 1.5 eq) in pyridine (10 mL, 0.14 M) was added EDCI (543 mg, 2.83 mmol, 2.0 eq). The reaction mixture was stirred at 25° C. for 2 h. The mixture was concentrated in vacuo, and the resulting crude residue was purified via silica gel chromatography (0-100% EtOAc in petroleum ether, followed by 0-10% MeOH in DCM) to yield the title compound as a brown solid. LCMS [M+H]⁺=383.1. ¹H NMR (400 MHz, CDCl₃) δ ppm: 10.88 (br s, 1H), 8.51-8.38 (m, 2H), 7.99 (d, J=8.8 Hz, 1H), 7.72-7.53 (m, 1H), 4.38 (br s, 1H), 4.19-4.10 (m, 1H), 3.84 (d, J=8.8 Hz, 3H), 3.81-3.75 (m, 2H), 3.64-3.49 (m, 2H), 3.47 (s, 1H), 2.20-1.99 (m, 3H), 1.32-1.28 (m, 3H), 1.27-1.25 (m, 1H).

ethyl 2-(8-cyano-1-((2R,4R)-2-methyltetrahydro-2H-pyran-4-yl)-1H-imidazo[4,5-c]quinolin-2-yl)acetate: To a solution of ethyl 3-((6-cyano-4-(((2R,4R)-2-methyltetrahydro-2H-pyran-4-yl)amino)quinolin-3-yl)amino)-3-oxopropanoate (500 mg, 941 μmol, 1 eq) in DMF (5 mL, 0.2 M) was added AcOH (0.5 mL, 8.74 mmol, 0.5 mL, 9.3 eq). The mixture was stirred at 130° C. for 9 h under microwave (2 bar). The reaction mixture was concentrated in vacuo, and the resulting crude material was purified via silica gel chromatography (50-100% EtOAc in petroleum ether) to yield the title compound as a brown solid. LCMS [M+H]⁺=365.1. ¹H NMR (400 MHz, CDCl₃) δ ppm: 9.43 (s, 1H), 9.10 (d, J=18.8 Hz, 1H), 8.66 (br s, 1H), 7.96 (d, J=8.8 Hz, 1H), 4.93 (br s, 1H), 4.37 (br s, 1H), 4.30 (s, 2H), 3.83 (s, 3H), 3.76 (br s, 2H), 2.41 (br s, 1H), 2.17 (br s, 1H), 2.02 (s, 2H), 1.40 (d, J=6.0 Hz, 3H).

2-(8-cyano-1-((2R,4R)-2-methyltetrahydro-2H-pyran-4-yl)-1H-imidazo[4,5-c]quinolin-2-yl)acetohydrazide: A solution of ethyl 2-(8-cyano-1-((2R,4R)-2-methyltetrahydro-2H-pyran-4-yl)-1H-imidazo[4,5-c]quinolin-2-yl)acetate (50 mg, 112 μmol, 1 eq), hydrazine hydrate (11.2 μL, 225 μmol, 2.0 eq) in EtOH (2 mL, 0.05 M) was stirred at 0° C. for 2 h. The reaction mixture was filtered, purified by prep HPLC (NH₄HCO₃ conditions) and lyophilized to yield the title compound as a white solid. LCMS [M+H]⁺=365.1. ¹H NMR (400 MHz, DMSO-d₆) δ ppm: 9.48 (br s, 1H), 9.33 (s, 1H), 9.04 (br s, 1H), 8.33 (d, J=8.4 Hz, 1H), 8.05 (dd, J=1.6, 8.4 Hz, 1H), 5.29 (br s, 1H), 4.35 (d, J=4.0 Hz, 2H), 4.20 (br s, 1H), 4.12 (br s, 2H), 3.74 (br s, 2H), 2.28-2.04 (m, 4H), 1.25 (d, J=6.0 Hz, 3H).

1-[(2R,4R)-2-methyloxan-4-yl]-2-[(5-oxo-4,5-dihydro-1,3,4-oxadiazol-2-yl)methyl]-1H-imidazo[4,5-c]quinoline-8-carbonitrile (Compound 7): To a solution of 2-(8-cyano-1-((2R,4R)-2-methyltetrahydro-2H-pyran-4-yl)-1H-imidazo[4,5-c]quinolin-2-yl)acetohydrazide (17 mg, 45.1 μmol, 1 eq) in DMSO (0.5 mL, 0.1 M) was added CDI (14.6 mg, 90.2 μmol, 2.0 eq). The mixture was stirred at 70° C. for 1 h under microwave (2 bar). The reaction mixture was filtered, purified by prep HPLC (formic acid conditions) and lyophilized to yield example 7 as a white solid. LCMS [M+H]⁺=391.2. ¹H NMR (400 MHz, DMSO-d₆) δ ppm: 12.37 (s, 1H), 9.40 (s, 1H), 9.02 (br s, 1H), 8.34 (d, J=8.4 Hz, 1H), 8.08 (dd, J=1.2, 8.8 Hz, 1H), 5.21 (br s, 1H), 4.71 (s, 2H), 4.18 (br s, 1H), 3.92-3.60 (m, 2H), 2.48-2.40 (m, 1H), 2.23-1.92 (m, 3H), 1.24 (d, J=6.0 Hz, 3H).

### Example 8. Procedure H: Synthesis of Compound 8

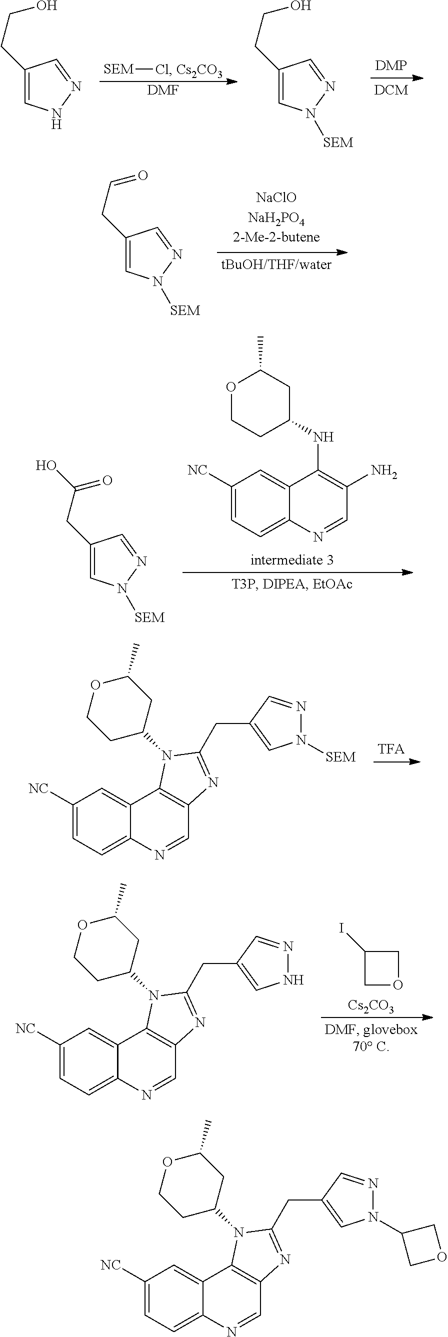

2-(1-((2-(trimethylsilyl)ethoxy)methyl)-1H-pyrazol-4-yl)ethan-1-ol: To a suspension of 2-(1H-pyrazol-4-yl)ethanol (5 g, 44.6 mmol, 1 eq) and Cs₂CO₃ (21.8 g, 66.9 mmol, 1.5 eq) in DMF (50 mL, 0.9 M) was added SEM-Cl (11.8 mL, 66.9 mmol, 1.5 eq) at 25° C. The reaction mixture was stirred at 25° C. for 2 h. The mixture was quenched with water (250 mL) and extracted with EtOAc (3×100 mL). The combined organic layers were washed with water (3×100 mL) and brine (100 mL). The organic layer was dried over Na₂SO₄, filtered, and concentrated in vacuo. The resulting crude material was purified via silica gel chromatography (30% EtOAc in petroleum ether) to yield the title compound as a colorless oil. LCMS [M+H]⁺=243.1. ¹H NMR (500 MHz, CDCl₃) δ ppm: 7.44 (s, 1H), 7.43 (s, 1H), 5.38 (s, 2H), 3.79 (t, J=6.5 Hz, 2H), 3.58-3.53 (m, 2H), 2.74 (t, J=6.5 Hz, 2H), 0.92-0.88 (m, 2H), −0.03 (s, 9H).

2-(1-((2-(trimethylsilyl)ethoxy)methyl)-1H-pyrazol-4-yl)acetaldehyde: A suspension of 2-(1-((2-(trimethylsilyl)ethoxy)methyl)-1H-pyrazol-4-yl)ethan-1-ol (4 g, 16.5 mmol, 1 eq), Dess-Martin periodinane (7.30 mL, 23.6 mmol, 1.43 eq) in DCM (50 mL) was stirred at 25° C. for 4 h. The reaction mixture was diluted with DCM (250 mL) and washed with saturated aq. NaHCO₃ (250 mL), water (250 mL) and brine (100 mL). The organic layer was dried over Na₂SO₄, filtered and concentrated in vacuo. The resulting crude material was purified via silica gel chromatography (20% EtOAc in petroleum ether) to yield the title compound as a yellow oil. LCMS [M+H]⁺=241.1. ¹H NMR (400 MHz, CDCl₃) 9.74 (t, J=1.6 Hz, 1H), 7.55 (s, 1H), 7.47 (s, 1H), 5.42-5.41 (m, 2H), 3.63-3.60 (m, 1H), 3.61 (d, J=1.5 Hz, 1H), 3.56 (d, J=8.0 Hz, 2H), 0.93-0.89 (m, 2H), −0.02 (s, 9H).

2-(1-((2-(trimethylsilyl)ethoxy)methyl)-1H-pyrazol-4-yl)acetic acid: To a solution of 2-(1-((2-(trimethylsilyl)ethoxy)methyl)-1H-pyrazol-4-yl)acetaldehyde (1 g, 4.16 mmol, 1 eq), 2-methylbut-2-ene (2.64 mL, 25.0 mmol, 6 eq) in t-BuOH (20 mL), THF (10 mL) and water (5 mL) was added NaH₂PO₄ (1.5 g, 12.5 mmol, 3 eq) and NaClO₂ (933 μL, 12.5 mmol, 3 eq) at 25° C. The reaction mixture was stirred at 25° C. for 1 h, then concentrated in vacuo. The residue was diluted with water (100 mL) and extracted with EtOAc (2×100 mL). The combined organic layers were washed with water (100 mL) and brine (100 mL). The organic layer was dried over Na₂SO₄, filtered, and concentrated in vacuo to yield the title compound as a colorless oil, which was used in the next step without further purification. LCMS [M+H]⁺=257.1.

1-((2R,4R)-2-methyltetrahydro-2H-pyran-4-yl)-2-((1-((2-(trimethylsilyl)ethoxy)methyl)-1H-pyrazol-4-yl)methyl)-1H-imidazo[4,5-c]quinoline-8-carbonitrile: A solution of 2-(1-((2-(trimethylsilyl)ethoxy)methyl)-1H-pyrazol-4-yl)acetic acid (600 mg, 2.34 mmol, 1.32 eq), intermediate 3 (500 mg, 1.77 mmol, 1 eq), DIPEA (1.8 g, 13.93 mmol, 2.43 mL, 7.86 eq) and T3P (50% in EtOAc, 1.40 mL, 4.71 mmol, 2.7 eq) in EtOAc (10 mL) was stirred at 80° C. under microwave irradiation for 6 h. The reaction mixture was diluted with EtOAc (50 mL) and washed with water (50 mL) and brine (50 mL). The organic layer was dried over Na₂SO₄, filtered, and concentrated in vacuo. The resulting crude material was purified via silica gel chromatography (100% EtOAc) to yield the title compound as a yellow oil. LCMS [M+H]⁺=503.2.

2-((1H-pyrazol-4-yl)methyl)-1-((2R,4R)-2-methyltetrahydro-2H-pyran-4-yl)-1H-imidazo[4,5-c]quinoline-8-carbonitrile: To a solution of 1-((2R,4R)-2-methyltetrahydro-2H-pyran-4-yl)-2-((1-((2-(trimethylsilyl)ethoxy)methyl)-1H-pyrazol-4-yl)methyl)-1H-imidazo[4,5-c]quinoline-8-carbonitrile (280 mg, 557 μmol, 1 eq) in DCM (10 mL) was added TFA (3 mL) at 25° C. The reaction mixture was stirred at 25° C. for 3 h. The reaction mixture was then concentrated in vacuo to afford the title compound as a yellow oil, which was used in next step without further purification. LCMS [M+H]⁺=373.1.

1-[(2R,4R)-2-methyloxan-4-yl]-2-{[1-(oxetan-3-yl)-1H-pyrazol-4-yl]methyl}-1H-imidazo[4,5-c]quinoline-8-carbonitrile (Compound 8): In a glove box, to a suspension of 2-((1H-pyrazol-4-yl)methyl)-1-((2R,4R)-2-methyltetrahydro-2H-pyran-4-yl)-1H-imidazo[4,5-c]quinoline-8-carbonitrile (80 mg, 214 μmol, 1 eq) and Cs₂CO₃ (160 mg, 491 μmol, 2.3 eq) in DMF (1 mL) was added 3-iodooxetane (160 mg, 869 μmol, 4.1 eq) at 25° C. The reaction mixture was stirred at 70° C. in a glove box for 4 h. The reaction mixture was then cooled to 25° C., diluted with MeOH (2 mL) and filtered. The filtrate was purified by prep-HPLC (NH₄HCO₃ conditions) to yield example 8 as a yellow solid. LCMS [M+H]⁺=429.1. ¹H NMR (400 MHz, DMSO-d₆) δ ppm: 9.35 (s, 1H), 8.99-8.95 (m, 1H), 8.33 (d, J=8.4 Hz, 1H), 8.06-8.02 (m, 1H), 7.83 (s, 1H), 7.57 (s, 1H), 5.54 (quin, J=7.2 Hz, 1H), 5.47-5.09 (m, 1H), 4.92-4.86 (m, 2H), 4.85-4.81 (m, 2H), 4.42 (s, 2H), 4.13 (br s, 1H), 3.73 (br s, 2H), 2.16-1.63 (m, 4H), 1.20 (d, J=6.0 Hz, 3H).

### Example 9. Synthesis of Additional Compounds of Formula (I)

Additional compounds of the present invention were synthesized according to the above Procedures as described in Table 3 below.

TABLE 3
Syntheses

| Compound | Procedure | Name |
|---|---|---|
| 9 | A | 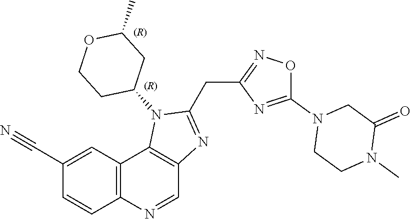 |
| | | 2-{[5-(4-methyl-3-oxopiperazin-1-yl)-1,2,4-oxadiazol-3- |
| | | yl]methyl}-1-[(2R,4R)-2-methyloxan-4-yl]-1H- |
| | | imidazo[4,5-c]quinoline-8-carbonitrile |
| 10 | E | 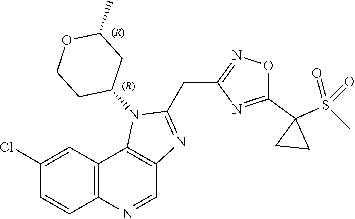 |
| | | 3-({8-chloro-1-[(2R,4R)-2-methyloxan-4-yl]-1H- |
| | | imidazo[4,5-c]quinolin-2-yl}methyl)-5-(1- |
| | | methanesulfonylcyclopropyl)-1,2,4-oxadiazole |
| 11 | A | 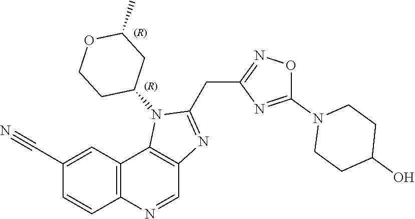 |
| | | 2-{[5-(4-hydroxypiperidin-1-yl)-1,2,4-oxadiazol-3- |
| | | yl]methyl}-1-[(2R,4R)-2-methyloxan-4-yl]-1H- |
| | | imidazo[4,5-c]quinoline-8-carbonitrile |
| 12 | A | 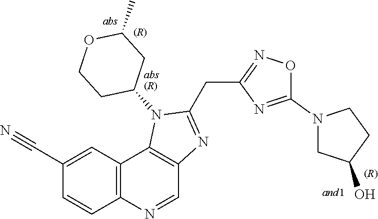 |
| | | 2-({5-[(3RS)-3-hydroxypyrrolidin-1-yl]-1,2,4-oxadiazol- |
| | | 3-yl}methyl)-1-[(2R,4R)-2-methyloxan-4-yl]-1H- |
| | | imidazo[4,5-c]quinoline-8-carbonitrile |
| 13 | E | 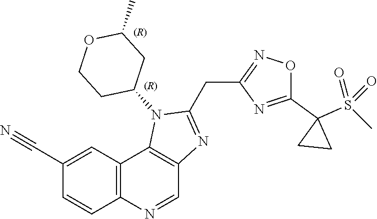 |
| | | 2-{[5-(1-methanesulfonylcyclopropyl)-1,2,4-oxadiazol-3- |
| | | yl]methyl}-1-[(2R,4R)-2-methyloxan-4-yl]-1H- |
| | | imidazo[4,5-c]quioline-8-carbonitrile |
| 14 | A | 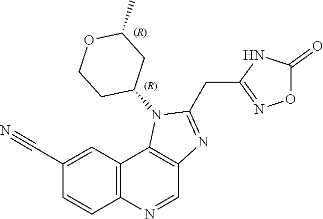 |
| | | 1-[(2R,4R)-2-methyloxan-4-yl]-2-[(5-oxo-4,5-dihydro- |
| | | 1,2,4-oxadiazol-3-yl)methyl]-1H-imidazo[4,5- |
| | | c]quinoline-8-carbonitrile |
| 15 | A | 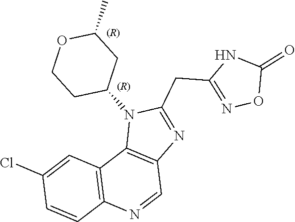 |
| | | 3-({8-chloro-1-[(2R,4R)-2-methyloxan-4-yl]-1H- |
| | | imidazo[4,5-c]quinolin-2-yl}methyl)-4,5-dihydro-1,2,4- |
| | | oxadiazol-5-one |
| 16 | D | 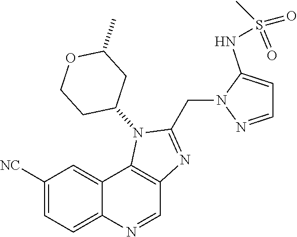 |
| | | N-(1-{[8-cyano-1-(2-methyloxan-4-yl)-1H-imidazo[4,5- |
| | | c]quinolin-2-yl]methyl}-1H-pyrazol-5- |
| | | yl)methanesulfonamide |
| 17 | B | 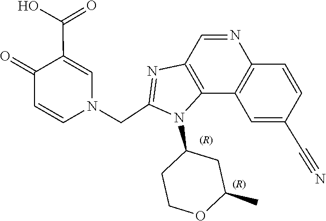 |
| | | 1-({8-cyano-1-[(2R,4R)-2-methyloxan-4-yl]-1H- |
| | | imidazo[4,5-c]quinolin-2-yl}methyl)-4-oxo-1,4- |
| | | dihydropyridine-3-carboxylic acid |
| 18 | B | 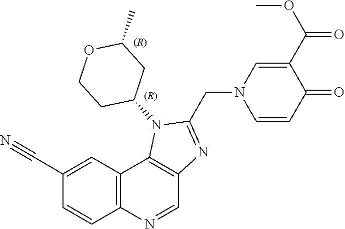 |
| | | methyl 1-({8-cyano-1-[(2R,4R)-2-methyloxan-4-yl]-1H- |
| | | imidazo[4,5-c]quinolin-2-yl}methyl)-4-oxo-1,4- |
| | | dihydropyridine-3-carboxylate |
| 19 | B | 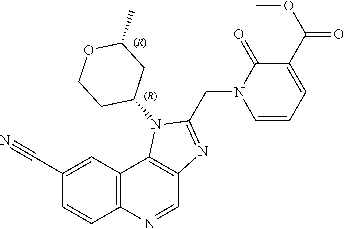 |
| | | methyl 1-({8-cyano-1-[(2R,4R)-2-methyloxan-4-yl]-1H- |
| | | imidazo[4,5-c]quinolin-2-yl}methyl)-2-oxo-1,2- |
| | | dihydropyridine-3-carboxylate |
| 20 | E | 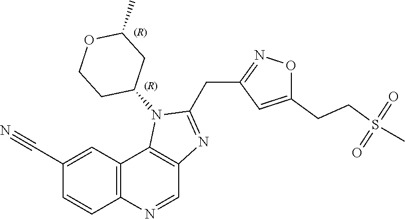 |
| | | 2-{[5-(2-methanesulfonylethyl)-1,2-oxazol-3-yl]methyl}- |
| | | 1-[(2R,4R)-2-methyloxan-4-yl]-1H-imidazo[4,5- |
| | | c]quinoline-8-carbonitrile |
| 21 | E | 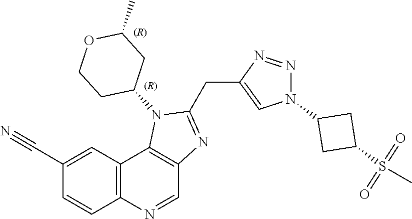 |
| | | 2-{1-(3-methanesulfonylcyclobutyl)-1H-1,2,3-triazol-4- |
| | | yl]methyl}-1-[(2R,4R)-2-methyloxan-4-yl]-1H- |
| | | imidazo[4,5-c]quinoline-8-carbonitrile |
| 22 | E | 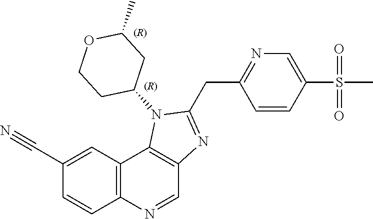 |
| | | 2-[(5-methanesulfonylpyridin-2-yl)methyl]-1-[(2R,4R)-2- |
| | | methyloxan-4-yl]-1H-imidazo[4,5-c]quinoline-8- |
| | | carbonitrile |

Characterization of illustrative compounds of the present disclosure are provided in Table 4.

TABLE 4
LCMS and NMR Data

| | LCMS | |
|---|---|---|
| Compound | [M + H]⁺ | NMR (in DMSO-d₆ unless otherwise indicated) |
| 9 | 487.3 | (400 MHz) δ 9.34 (s, 1H), 9.03 (s, 1H), 8.34 (d, J = 8.8 Hz, 1H), |
| | | 8.06 (d, J = 9.6 Hz, 1H), 5.44-5.25 (m, 1H), 4.64-4.54 (m, 2H), |
| | | 4.26-4.14 (m, 1H), 4.06 (s, 2H), 3.79-3.64 (m, 4H), 3.45-3.41 (m, |
| | | 2H), 2.88-2.86 (m, 3H), 2.57-2.53 (m, 1H), 2.25-2.14 (m, 2H), |
| | | 2.12-2.01 (m, 1H), 1.24(d, J = 6.4 Hz, 3H) |
| 10 | 502.1 | (400 MHz) δ9.19(s, 1H), 8.80-8.60(m, 1H), 8.21(d, J = 9.2 Hz, 1H), |
| | | 7.77(dd, J = 2.0, 9.2 Hz, 1H), 5.43-5.15(m, 1H), 4.86(s, 2H), 4.25- |
| | | 4.10(m, 1H), 3.79-3.56(m, 2H), 3.32(s, 3H), 2.47-2.40(m, 1H), |
| | | 2.24-2.00 (m, 3H), 1.94-1.88(m, 2H), 1.87-1.79 (m, 2H), 1.24(d, J = |
| | | 6.4 Hz, 3H) |
| 11 | 474.2 | (400 MHz) δ 9.35 (s, 1H), 9.02 (s, 1H), 8.34 (d, J = 8.4 Hz, 1H), |
| | | 8.06 (d, J = 8.4 Hz, 1H), 5.43-5.24 (m, 1H), 4.85 (d, J = 4.0 Hz, |
| | | 1H), 4.55 (s, 2H), 4.26-4.13 (m, 1H), 3.78-3.66 (m, 5H), 3.31-3.27 |
| | | (m, 2H), 2.56-2.52 (m, 1H), 2.23-2.11 (m, 2H), 2.09-1.98 (m, 1H), |
| | | 1.82-1.72 (m, 2H), 1.44-1.34 (m, 2H), 1.23 (d, J = 6.0 Hz, 3H) |
| 12 | 460.2 | (400 MHz) δ 9.35 (s, 1H), 9.03 (s, 1H), 8.34 (d, J = 8.4 Hz, 1H), |
| | | 8.06 (dd, J = 1.6, 8.4 Hz, 1H), 5.45-5.26 (m, 1H), 5.15-5.05 |
| | | (m, 1H), 4.55 (s, 2H), 4.39-4.32 (m, 1H), 4.26-4.12 (m, 1H), 3.84- |
| | | 3.61 (m, 2H), 3.57-3.48 (m, 3H), 3.28-3.27 (m, 1H), 2.48-2.42 (m, |
| | | 1H), 2.27-2.15 (m, 2H), 2.12-2.04 (m, 1H), 2.02-1.94 (m, 1H), |
| | | 1.92-1.84 (m, 1H), 1.24 (d, J = 6.0 Hz, 3H) |
| 13 | 493.1 | (400 MHz)δ9.34(s, 1H), 9.03(br s, 1H), 8.34(d, J = 8.8 Hz, 1H), 8.10- |
| | | 8.03(m, 1H), 5.45-5.25(m, 1H), 4.88(s, 2H), 4.25-4.13(m, 1H), |
| | | 3.80-3.62(m, 2H), 3.33-3.31 (m, 3H), 2.25-1.96(m, 4H), 1.95-1.88 |
| | | (m, 2H), 1.87-1.80(m, 2H), 1.24(d, J = 6.0 Hz, 3H) |
| 14 | 391.2 | (400 MHz)δ9.39 (s, 1H), 9.01 (br s, 1H), 8.34 (d, J = 8.8Hz, 1H), |
| | | 8.07 (dd, J = 1.6, 8.8Hz, 1H), 5.25 (br s, 1H), 4.61 (s, 2H), 4.17 (br |
| | | s, 1H), 3.87-3.63 (m, 2H), 2.43-2.38 (m, 1H), 2.23-1.96 (m, 3H), |
| | | 1.24 (d, J = 6.0 Hz, 3H) |
| 15 | 400.1 | (400 MHz)δ9.24 (s, 1H), 8.79-8.49 (m, 1H), 8.20 (d, J = 8.8Hz, |
| | | 1H), 7.76 (dd, J = 2.0, 8.8Hz, 1H), 7.07 (br s, 1H), 5.17 (br s, 1H), |
| | | 4.56 (s, 2H), 4.17 (br s, 1H), 3.83-3.57 (m, 2H), 2.46-2.43 (m, |
| | | 1H), 2.15 (br s, 2H), 2.04 (brs, 1H), 1.24 (d, J = 6.0 Hz, 3H) |
| 16 | 466.1 | (500 MHz) 8 9.36 (br s, 1H), 8.95 (s, 1H), 8.33 (d, J = 8.5 Hz, 1H), |
| | | 8.05 (d, J = 8.5 Hz, 1H), 7.27 (brs, 1H), 7.10 (br s, 1H), 5.95 (br |
| | | s, 1H), 5.79-5.61 (m, 2H), 5.45-5.28 (m, 1H), 4.09 (d, J = 6.5 Hz, |
| | | 1H), 3.84-3.58 (m, 2H), 2.90 (br s, 3H), 2.41-2.31 (m, 1H), 2.12- |
| | | 2.03 (m, 1H), 1.89 -1.72 (m, 1H), 1.67-1.48 (m, 1H), 1.19 (d, J = |
| | | 6.0 Hz, 3H) |
| 17 | 444.2 | (400 MHz)δ9.32 (s, 1H), 9.08-8.89 (m, 2H), 8.32 (d, J = 8.8 Hz, |
| | | 1H), 8.25 (dd, J = 2.0, 7.6 Hz, 1H), 8.08 (dd, J = 1.6, 8.8Hz, 1H), |
| | | 6.89 (d, J = 7.2 Hz, 1H), 6.08 (s, 2H), 5.77-4.94 (m, 1H), 4.27-4.11 |
| | | (m, 1H), 3.96-3.71 (m, 2H), 2.47-2.35 (m, 1H), 2.31-2.09 (m, 3H), |
| | | 1.27 (d, J = 6.0 Hz, 3H) |
| 18 | 458.2 | (400 MHz)δ9.36 (s, 1H), 8.97 (br s, 1H), 8.55 (d, J = 2.4 Hz, 1H), |
| | | 8.33 (d, J = 8.8 Hz, 1H), 8.10-8.06 (m, 1H), 7.78 (dd, J = 2.4, 7.6 |
| | | Hz, 1H), 6.33 (d, J = 7.6 Hz, 1H), 5.86 (s, 2H), 4.17 (br s, 1H), |
| | | 3.84 (br s, 2H), 3.71 (s, 3H), 3.42-3.33 (m, 1H), 2.43 (br s, 1H), |
| | | 2.30-2.04 (m, 3H), 1.26 (d, J = 6.4 Hz, 3H) |
| 19 | 458.2 | (400 MHz)δ9.28 (s, 1H), 9.01 (s, 1H), 8.31 (d, J = 8.4Hz, 1H), 8.22 |
| | | (dd, J = 2.0, 6.8Hz, 1H), 8.15 (dd, J = 2.0, 7.2Hz, 1H), 8.05 (dd, J = |
| | | 1.6, 8.4 Hz, 1H), 6.49 (t, J = 6.8 Hz, 1H), 5.69 (s, 2H), 5.60-5.41 |
| | | (m, 1H), 4.23-4.10 (m, 1H), 3.94-3.75 (m, 2H), 3.71 (s, 3H), 2.47- |
| | | 2.36 (m, 1H), 2.30-2.05 (m, 3H), 1.27 (d, J = 6.4Hz, 3H) |
| 20 | 480.2 | (400 MHz) δ 9.37 (s, 1H), 9.01 (br s, 1H), 8.34 (d, J -8.8 Hz, 1H), |
| | | 8.07 (dd, J = 1.6, 8.8 Hz, 1H), 6.43 (s, 1H), 5.25 (br s, 1H), 4.68 (br |
| | | s, 2H), 4.25-4.09 (m, 1H), 3.83-3.59 (m, 2H), 3.54 (t, J = 8.0 Hz, |
| | | 2H), 3.23 (t, J = 8.0 Hz, 2H), 3.02 (s, 3H), 2.32-1.78 (m, 4H), 1.23 |
| | | (d, J = 6.0 Hz, 3H) |
| 21 | 506.4 | (400 MHz) δ 9.62 (s, 1H), 9.09 (s, 1H), 8.49 (d, J = 8.8 Hz, 1H), |
| | | 8.27-8.14 (m, 2H), 5.44 (br s, 1H), 5.24-5.15(m, 1H), 4.74 (s, 2H), |
| | | 4.16 (br s, 1H), 3.95 (d, J = 9.2Hz, 2H), 2.96 (s, 3H), 2.91-2.76 (m, |
| | | 4H), 2.48-2.37 (m, 2H), 2.24-1.85 (m, 3H), 1.23 (d, J = 6.0 Hz, 3H) |
| 22 | 462.2 | (400 MHz, CDCl3) δ 9.38 (s, 1H), 9.08 (d, J = 2.0 Hz, 2H), 8.39 |
| | | (d, J = 8.4 Hz, 1H), 8.23 (dd, J = 2.4, 8.4 Hz, 1H), 7.87 (dd, J = 1.2, |
| | | 8.8 Hz, 1H), 7.66 (d, J = 8.0 Hz, 1H), 5.40-5.28 (m, 1H), 4.81 (s, |
| | | 2H), 4.38-4.28 (m, 1H), 3.81-3.62 (m, 2H), 3.11 (s, 3H), 2.76-2.55 |
| | | (m, 1H), 2.47-2.19 (m, 1H), 2.13-1.78 (m, 2H), 1.37 (d, J = 6.0 Hz, |
| | | 3H) |

### Example 10. Biological Activity

Biochemical Assay

Materials:
LRRK2 wild type enzyme
Substrate (LRRKtide)
ATP
TR-FRET dilution buffer
pLRRKtide antibody
384-well assay plate
DMSO
Enzyme reaction conditions
50 mM Tris pH 8.0, 5 mM MgCl₂, 1 mM EDTA, 0.01% Brij-35, 2 mM DTT.
3 nM LRRK2
400 nM LRRKtide
25 μM ATP
120 minutes reaction time
23° C. reaction temperature
10 μL total reaction volume
Detection reaction conditions
TR-FRET dilution buffer
10 mM EDTA
0.25 nM antibody
23° C. reaction temperature
10 μL detection reagent was added and 20 μL volume in total in each well

10 mM compound solution was prepared in DMSO. An 11-point, 3-fold dilution was performed, with highest concentration at 10 μM. A 10 mM compound DMSO solution was added to Labcyte LDV plate and the compound concentration of source plate was 10 mM. The 1 st compound concentration of Inter plate (Labcyte 384 well PP plate) was 4.938×10⁻¹ mM which was prepared by transferring 1.5 μL of 10 mM compound from source plate to 28.9 μL DMSO. The 2nd compound concentration of Inter plate was 1.829×10⁻² mM which was prepared by transferring 60 nL of 10 mM compound from source plate to 32.7 μL DMSO. The 3rd compound concentration of Inter plate was 6.774×10⁻⁴ mM which was prepared by transferring 2.5 nL of 10 mM compound from source plate to 36.9 μL DMSO. 100 nL of reference compound was dispensed in column 1 for low control wells and 100 nL of DMSO in column 24 for high control wells. Compounds were dispensed to column 2 to 23 of assay plate and backfilled with DMSO to a total volume of 100 nL.

A 2×LRRK2 enzyme solution (final concentration 3 nM) was prepared in assay buffer (Tris-HCl pH8.0: 50 mM, MgCl₂: 5 mM, EDTA: 1 mM, Brij-35: 0.01%, 2 mM DTT). A 2× substrate solution was prepared: LRRK2 tide substrate (final concentration 400 nM) and ATP (final concentration 25 μM) in assay buffer. 5 μL 2×LRRK2 enzyme solution was dispensed to each well in assay plate by Multidrop. Assay plate was spun at 1,000 rpm for 1 min and incubated for 15 min at 23° C. 5 μL 2×ATP/LRRKtide solution was dispensed to each well in assay plate by Multidrop. Assay plate was spun at 1,000 rpm for 1 min and incubated for 120 min at 23° C.

A 2× detection solution was prepared: Tb-pERM (pLRRKtide) antibody (final concentration 0.25 nM) and EDTA (final concentration 10 mM) in TR-FRET dilution buffer. 10 μL 2× detection solution was dispensed to each well in assay plate to stop kinase reaction by Multidrop. Assay plate was spun at 1,000 rpm for 1 min and incubate for 30 min at 23° C. Assay plate was then read on Envision configured for LanthaScreen® TR-FRET.

pS935 LRRK2 Cell Assay

The following protocol describes an in-vitro method for measuring phosphorylation at Ser935 on wild type LRRK2 overexpressed in recombinant HEK-293T cells. The method is based on HTRF technology, which combines Fluorescence Resonance Energy Transfer (FRET) with Time-Resolved measurement (TR). Phospho-LRRK2 (Ser935) is detected in a sandwich assay format using two different specific antibodies, one labelled with Eu³⁺-Cryptate (donor) and the second with d2 (acceptor). When the fluorophores are in close proximity, excitation of the donor with a light source (flash lamp) stimulates FRET to the acceptor, which in turn fluoresces at a specific wavelength (665 nm). The fluorescence emission at 615 nm from the donor is also measured to allow ratiometric reduction of data. The specific signal is proportional to phospho-LRRK2 (Ser935).

TABLE 5
Materials for Cell Assay

| Reagent | Source | Catalog No. |
|---|---|---|
| pCMV-LRRK2 WT | WuXi | N/A |
| plasmid | | |
| pCMV-LRRK2 G2019S | WuXi | N/A |
| plasmid | | |
| Fetal bovine serum(FBS) | Corning | 35-081-CV |
| TransIT-LT1 transfection | Mirus | 2305 |
| reagent | | |
| DMEM (1X) + | Gibco | 11965-092 |
| GLUTAMAX | | |
| 0.25% Trypsin -EDTA(1X) | Gibco | 25200-072 |
| DPBS (1X) | Gibco | 14190-144 |
| LRRK2 phospho-S935 kit | Cisbio | 6FLRKPEH- |
| | | 05G |
| DMSO | SIGMA | 472301 |

### Protocol:

### Day 0:

Plasmid transient transfection: DMEM medium, FBS, DPBS, Trans-IT, OPTI-MEM reagents were allowed to warm to room temperature. HEK293T cells were cultured in DMEM+10% FBS complete medium in T150 flasks till around 80% confluency before transfection. Then cells were washed with 10 mL PBS and detached with 3 mL 0.25% typinsin. 30×10E6 HEK293T cells were seeded in DMEM+10% FBS complete medium to a 15 cm dish.

DNA, TransIT-LT1, OPTI-MEM complex were prepared: 2000 L OPTI-MEM was added to a 15 mL cone tube, then 20 g plasmid was added to OPTI-MEM and mixed, followed by addition of 60 L TransIT-LT1 to the plasmid OPTI-MEM mixture and mixing. The resulting mixture was incubated for 15 minutes.

The above plasmid, DNA and OPTI-MEM mixture was added dropwise to the 15 cm dish, ensuring the drops were evenly distributed. The culture dish was gently rocked back and forth and from left to right to evenly distribute the complex. The transfected dish was incubated for 24 hours at 37° C., and 5% CO₂.

### Day 1:

Transfected HEK293T was collected in the 15 cm dish. Media was aspirated from tissue culture dish, and washed by dispensing 10 mL 1×DPBS into 15 cm dish. 1×DPBS was aspirated and 3 mL trypsin was dispensed to 15 cm dish. The dish was incubated with trypsin at room temperature for 3 min until the cells were detached. 10 mL of DMEM+10% FBS medium were added to 15 cm dish and triturated to ensure homogenous cell suspensions.

Homogenous cell suspensions were transferred to a 50 mL tube, and centrifuged for 5 minutes at 1,000 rmp/min. The supernatant was aspirated and resuspended with 20 mL complete medium. 1 mL of cell suspension was transferred for cell counting. The cell suspension was diluted to 2×10E5 cells/ml. 50 L cell suspensions were added to a 384-well plate. The plate was quick spun at 800 rpm for 1 minute, then incubate overnight at 37° C., 5% CO₂.

### Day 2:

Compound dispensing: compound was diluted (10 mM DMSO stock solution) and added to assay plate (top concentration: 10 μM, 3 fold serial dilution, 9 doses) with duplicates by Tecan liquid handler. The DMSO concentration of each well was normalized to 0.2%. Plates were quick spun at 1,000 rpm for 1 minute. Plates were incubated at 37° C., 5% CO₂ for 2 hours.

1× lysis buffer solution supplemented with blocking reagent was prepared (e.g. 1 mL lysis buffer 4×+3 mL water+40 L stock blocking reagent 100×). Antibodies working solution was prepared by diluting 40-fold d2 and Cryptate antibodies with detection buffer (e.g. 1520 L detection buffer+40 L d2-antibody stock solution+40 L Cryptate-antibody stock solution).

After 2 hours incubation, cell plates were taken out from the incubator. Medium was removed by plate washer, then 16 L of supplemented lysis buffer was added 1× to each well and incubated for 30 minutes at room temperature under shaking (800 rpm/min). 4 L of antibodies working solution was added to each well, covered with a top seal and incubated overnight in 23° C. incubator.

### Day 3:

HTRF signal was read (665 nm and 615 nm) on Wallac 2104 EnVision® multilabel reader. Data were analyzed by XL fit software.

Biochemical data for illustrative compounds of the present disclosure are shown in Table 6.

TABLE 6
LRRK2 Inhibition

| | Biochemical | Cell IC₅₀ |
|---|---|---|
| Compound | IC₅₀ (nM) | (nM) |
| 1 | 0.976 | 664 |
| 2 | 106 | |
| 3 | 0.798 | 61.5 |
| 4 | 0.888 | 458 |
| 5 | 0.635 | 324 |
| 6 | 3.94 | 4380 |
| 7 | 1.49 | 466 |
| 8 | 6.47 | 618 |
| 9 | 1.69 | 616 |
| 10 | 0.299 | 10.3 |
| 11 | 2.26 | 270 |
| 12 | 1.36 | 811 |
| 13 | 0.449 | 23.7 |
| 14 | 7.84 | >10000 |
| 15 | 3.23 | >10000 |
| 16 | 671 | |
| 17 | 2.88 | 2950 |
| 18 | 2.64 | 7020 |
| 19 | 8.03 | 592 |
| 20 | 0.596 | |
| 21 | 1.41 | 1770 |
| 22 | 0.579 | |

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A compound of Formula (I): 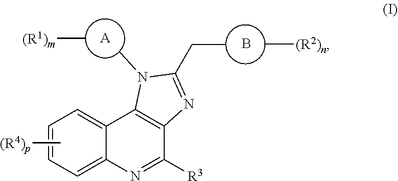
or a pharmaceutically acceptable salt thereof, wherein
Ring A is a 5 to 6 membered heterocycloalkyl having 1 to 2 heteroatoms each independently N, O or S, or a 5 to 6 membered heteroaryl having 1 or 2 heteroatoms each independently N, O or S;
each R¹ is independently C₁₋₆ alkyl or ═O;
Ring B is a 5 to 6 membered heteroaryl having 1 to 4 heteroatoms each independently N, O or S;
each R² is a C₁₋₆ alkyl, —OH, ═O, C(O)R^{2a}, C(O)OR^{2b}, OC(O)R^{2a}, S(O)₂R^{2a}, S(O)₂OR^{2b}, OS(O)₂R^{2a}, N(R^{2b})S(O)₂R^{2a}, S(O)₂N(R^{2b})(R^{2c}), C₃₋₆ cycloalkyl, C₁₋₆ alkyl-C₃₋₆ cycloalkyl, or 3 to 6 membered heterocycloalkyl having 1 to 3 heteroatoms each independently N, O or S, wherein each alkyl or cycloalkyl is substituted with 1 to 3 R^{2d} groups, and wherein each heterocycloalkyl is substituted with 0 to 3 R^{2e} groups;
each R^{2b} and R^{2c} is hydrogen or C₁₋₆ alkyl;
each R^{2d} is independently C(O)R^{2d1} or S(O)₂R^{2d1};
each R^{2e} is independently C₁₋₆ alkyl, —OH, ═O, C(O)R^{2e1} or S(O)₂R^{2e1};
each R^{2a}, R^{2d1} and R^{2e1} is independently C₁₋₆ alkyl;
each R³ and R⁴ is hydrogen, C₁₋₆ alkyl, C₁₋₆ alkoxy, halogen, C₁₋₆ haloalkyl, C₁₋₆ haloalkoxy, or —CN;
subscript n is 0, 1 or 2; and
subscript m and p are each independently an integer from 1 to 4.

2. The compound of claim 1, or a pharmaceutically acceptable salt thereof, wherein subscript n is 1 or 2.

3. The compound of claim 1 or 2, or a pharmaceutically acceptable salt thereof, wherein subscript m is 1.

4. The compound of any one of claims 1 to 3, or a pharmaceutically acceptable salt thereof, wherein subscript p is 1.

5. The compound of any one of claims 1 to 4, or a pharmaceutically acceptable salt thereof, wherein Ring A is a 5 to 6 membered heterocycloalkyl having 1 heteroatom of N, O or S, or a 5 to 6 membered heteroaryl having 1 or 2 heteroatoms each independently N, O or S.

6. The compound of any one of claims 1 to 5, or a pharmaceutically acceptable salt thereof, wherein Ring A is tetrahydropyranyl.

7. The compound of any one of claims 1 to 6, or a pharmaceutically acceptable salt thereof, wherein each R¹ is independently C₁₋₆ alkyl.

8. The compound of any one of claims 1 to 7, or a pharmaceutically acceptable salt thereof, wherein each R¹ is Me.

9. The compound of any one of claims 1 to 8, or a pharmaceutically acceptable salt thereof, wherein R³ is hydrogen or halogen.

10. The compound of any one of claims 1 to 9, or a pharmaceutically acceptable salt thereof, wherein R³ is hydrogen.

11. The compound of any one of claims 1 to 10, or a pharmaceutically acceptable salt thereof, wherein each R⁴ is independently halogen or —CN.

12. The compound of any one of claims 1 to 11, or a pharmaceutically acceptable salt thereof, wherein each R⁴ is independently Cl or —CN.

13. The compound of any one of claims 1 to 12, or a pharmaceutically acceptable salt thereof, having the structure of Formula Ia:

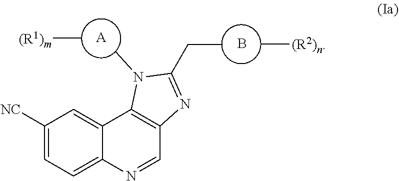

14. The compound of any one of claims 1 to 13, or a pharmaceutically acceptable salt thereof, having the structure of Formula Ib:

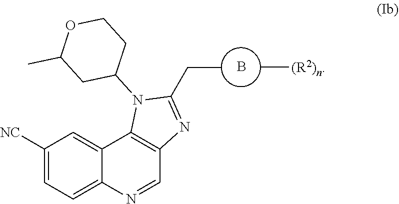

15. The compound of any one of claims 1 to 14, or a pharmaceutically acceptable salt thereof, having the structure of Formula Ic:

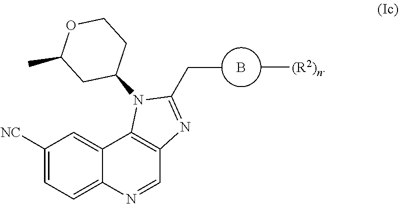

16. The compound of any one of claims 1 to 15, or a pharmaceutically acceptable salt thereof, wherein Ring B is a 5 to 6 membered heteroaryl having 1 to 3 heteroatoms each independently N, O or S.

17. The compound of any one of claims 1 to 16, or a pharmaceutically acceptable salt thereof, wherein Ring B is pyrazolyl, isoxazolyl, 1,2,3-triazolyl, 1,2,4-oxadiazolyl, 1,3,4-oxadiazolyl, or pyridyl.

18. The compound of any one of claims 1 to 17, or a pharmaceutically acceptable salt thereof, wherein
the group 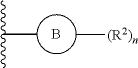
is

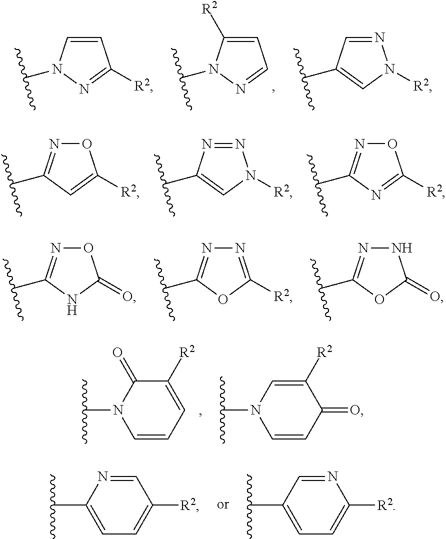

19. The compound of any one of claims 1 to 18, or a pharmaceutically acceptable salt thereof, wherein
each R² is C₁₋₃ alkyl, ═O, C(O)OR^{2b}, OC(O)R^{2a}, S(O)₂R^{2a}, N(R^{2b})S(O)₂R^{2a}, S(O)₂N(R^{2b})(R^{2c}), C₃₋₆ cycloalkyl, or 3 to 6 membered heterocycloalkyl having 1 to 3 heteroatoms each independently N, O or S, wherein each alkyl or cycloalkyl is substituted with 1 to 2 R^{2d} groups, and wherein each heterocycloalkyl is optionally substituted with 1 to 2 R^{2e} groups;
each R^{2b} and R^{2c} is hydrogen or C₁₋₃ alkyl;
each R^{2d} is independently C(O)R^{2d1} or S(O)₂R^{2d1};
each R^{2e} is independently C₁₋₃ alkyl, —OH, ═O, C(O)R^{2e1} or S(O)₂R^{2e1}; and
each R^{2a}, R^{2d1} and R^{2e1} is independently C₁₋₃ alkyl.

20. The compound of any one of claims 1 to 19, or a pharmaceutically acceptable salt thereof, wherein each R² is independently ═O, —COOH, —C(O)OMe, —SO₂Me, —NHSO₂Me, —CH₂CH₂SO₂Me,

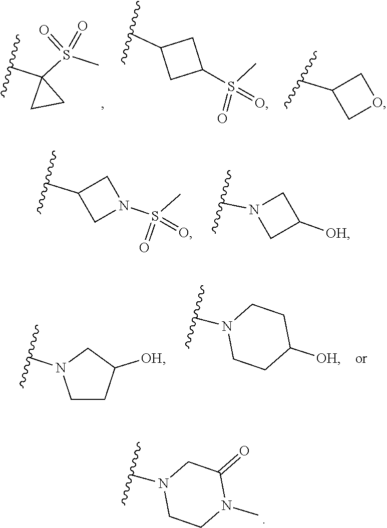

21. The compound of any one of claims 1 to 20, or a pharmaceutically acceptable salt thereof, wherein
the group 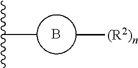
is

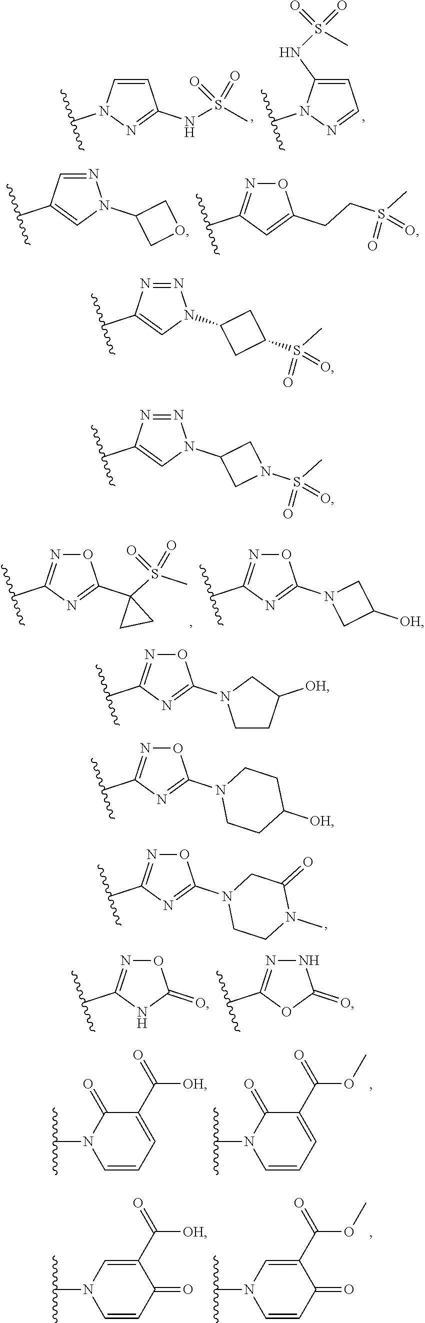

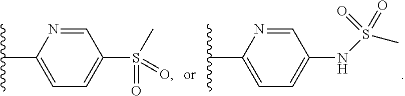 [SMILES: *c1ccc(S(C)(=O)=O)cn1 ; *c1ccc(NS(C)(=O)=O)cn1]

22. The compound of any one of claims 1 to 21, or a pharmaceutically acceptable salt thereof, wherein the compound has the structure of a compound in Table 1.

23. A pharmaceutical composition comprising a compound of any one of claims 1 to 22, or a pharmaceutically acceptable salt thereof, and a pharmaceutically acceptable excipient.

24. A method of inhibiting LRRK2 in a cell, the method comprising contacting the cell with an effective amount of a compound of any one of claims 1 to 22, or a pharmaceutically acceptable salt thereof.

25. A method of treating a LRRK2-associated disease or condition in a subject in need thereof, the method comprising administering to the subject a therapeutically effective amount of a compound of any one of claims 1 to 22, or a pharmaceutically acceptable salt thereof.

26. The method of claim 25, wherein the LRRK2-associated disease or condition is Parkinson's disease, Lewy body dementia, frontotemporal dementia, corticobasal dementia, progressive supranuclear palsy, Alzheimer's disease, tauopathy disease, or alpha-synucleinopathy.

27. The method of claim 25, wherein the LRRK2-associated disease or condition is an inflammatory bowel disease.

28. The method of claim 25, wherein the LRRK2-associated disease or condition is an autophagy-related disease or condition.

29. The method of claim 28, wherein the autophagy-related disease or condition is alpha 1-antitrypsin deficiency (AATD).
